SEQUENCE LISTING (3 sequences)

SEQ ID NO: 1 - protein, 484 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYNYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVK

SEQ ID NO: 2 - protein, 1251 aa, synthetic construct
MGRLASRPLLLALLSLALCRGRVVRYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVGGGGSGGGGSGGGGSGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLGGGGSGGGGSGGGGSSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTLLIGVNLLTVIGLWLFLILYC

SEQ ID NO: 3 - protein, 1251 aa, synthetic construct
MGRLASRPLLLALLSLALCRGRVVRNLDVEEPTIFQEDAGGFGQSVVQFGGSRLVVGAPLEVVAANQTGRLYDCAAATGMCQPIPLHIRPEAVNMSLGLTLAASTNGSRLLACGPTLHRVCGENSYSKGSCLLLGSRWEIIQTVPDATPECPHQEMGGGGSGGGGSGGGGSGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLGGGGSGGGGSGGGGSQSRASSSFQHEMSQEGFSTALTMDGLFLGAVGSFSWSGGAFLYPPNMSPTFINMSQENVDMRDSYLGYSTELALWKGVQNLVLGAPRYQHTGKAVIFTQVSRQWRKKAEVTGTQIGSYFGASLCSVDVDSDGSTDLILIGAPHYYEQTRGGQVSVCPLPRGRVQWQCDAVLRGEQGHPWGRFGAALTVLGDVNEDKLIDVAIGAPGEQENRGAVYLFHGASESGISPSHSQRIASSQLSPRLQYFGQALSGGQDLTQDGLMDLAVGARGQVLLLAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTLLIGVNLLTVIGLWLFLILYC

(19) United States
(12) Patent Application Publication
(10) Pub. No.: US 2026/0355351 A1
(43) Pub. Date: Nov. 12, 2026

(54) METHOD FOR EFFICIENTLY LOADING DNA INTO EXOSOME

(71) Applicant: Glycaliem Healthtech Co. Ltd., Beijing (CN)

(72) Inventors: Yi WANG, Beijing (CN); Haifeng SONG, Beijing (CN); Yanfang LI, Beijing (CN); Miaomiao XUE, Beijing (CN); Yanan DONG, Beijing (CN); Danfeng WANG, Beijing (CN)

(73) Assignee: Glycaliem Healthtech Co. Ltd., Beijing (CN)

(21) Appl. No.: 19/116,384

(22) PCT Filed: Sep. 30, 2022
(86) PCT No.: PCT/CN2022/123200

§ 371 Date: Mar. 28, 2025

(51) Int. Cl. A61K 48/00; C07K 14/705; C12N 9/22; C12N 15/10; C12N 15/11; C12N 15/87; C12Q 1/48; C12Q 1/6813; C12Q 1/6851; C12Q 1/686; C12Q 1/6876

(52) U.S. Cl. CPC A61K 48/0041; A61K 48/0091; C07K 14/70546; C12N 9/22; C12N 15/101; C12N 15/11; C12N 15/87; C12Q 1/48; C12Q 1/6813; C12Q 1/6851; C12Q 1/686; C12Q 1/6876; C12Y 207/07; C12Y 301/21001; C12Y 301/30002

(57) ABSTRACT

Provided is a method for efficiently loading DNA into an exosome, wherein the exosome is derived from mammalian cells or milk. A highly compressed DNA-polycationic compression agent complex is formed by combining a polycationic compression agent with a DNA, and the DNA-polycationic compression agent complex is loaded into the membrane of the exosome by means of a physical measures, wherein the cationic compression agent may be selected from one or more of protamine, polylysine, polyarginine, and polyethyleneimine; therefore, efficient in vivo gene delivery and expression of large-fragment DNA are smoothly realized by utilizing the transportation function of the exosome. The exosome obtained by the method is expected to become an efficient tool for loading, transporting, and expressing large-fragment DNA in the field of gene therapy.

### TECHNICAL FIELD

The present disclosure relates to the technical field of exosome loading, and in particular to a method for efficiently loading DNA into exosomes. The exosomes are derived from mammalian cells or milk. A highly compressed DNA-polycationic compression agent complex is formed by combining a polycationic compression agent with DNA, and then loaded into the membrane of the exosomes using physical measures, successfully achieving efficient loading of large-fragment DNA molecules as well as their in vivo gene delivery and expression.

### BACKGROUND OF RELATED ART

Gene therapy is an effective means to tackle genetic diseases, malignant tumors and some degenerative diseases. However, DNA-mediated gene therapy still faces many problems that are difficult to solve effectively: (1) DNA needs to enter not only the cell, but also the nucleus to be transcribed and expressed; and 2) a functionally complete DNA sequence requires multiple functional elements, including a promoter, target fragment, poly-A tail, and the like, resulting in sizes that are often much larger than those of therapeutic mRNA fragments. Currently, the mainstream DNA-based gene therapy methods still rely on adeno-associated virus (AAV). However, AAV vectors also have their own serious disadvantages, the most prominent of which being their safety risks in the human body. Clinical trials involving AAV-based gene therapy have repeatedly reported patient fatalities, and due to their immunogenicity, AAV drugs can only be administered as a single treatment, with no guarantee of lasting efficacy. Therefore, there is an urgent need for a non-viral delivery vector that can deliver DNA efficiently in vivo in the field of gene therapy.

Exosomes, a type of extracellular vesicles (EVs), has a natural function of delivering nucleic acid. They can be engineered for tissue and cell-specific delivery. As drug delivery vehicles, they have the outstanding advantages such as low immunogenicity, high transport efficiency, good stability, and strong targeting properties, as well as the ability to across the blood-brain barrier. If utilized in the field of DNA delivery, exosomes could serve as superior gene therapy vehicles compared to AAV.

At present, methods and technologies for loading RNA (such as mRNA and siRNA) into exosomes are relatively mature, whereas common methods like incubation or electroporation for loading RNA (such as mRNA and siRNA) into exosomes are only suitable for small-fragment nucleic acids such as miRNA, siRNA, and ASO, and are ineffective for mRNA or even large-fragment DNA or DNA plasmids. Regarding the demand for loading large DNA fragments into exosomes, there are a number of technical problems that are difficult to break through, including the large size of DNA fragments, difficulty in loading, and low efficiency of DNA entry into nucleus. In existing technologies, there is a method of achieving the loading of large-fragment nucleic acids by applying electrical stimulation to single cells placed in an electric field (https://doi.org/10.1038/s41551-019-0485-1). However, this method is difficult to achieve large-scale industrial applications and is limited to adherent cells. Additionally, the method developed by Carmine for loading plasmid DNA into extracellular vesicles from red blood cells (RBC exosomes) (WO2021/145821 A1) is limited to red blood cells-derived exosomes. Moreover, the required DNA loading reagents are inaccessible in many countries worldwide, greatly limiting the application and advancement of this technology.

Therefore, in view of the above technical difficulties in the field, the present disclosure provides a method for efficiently loading DNA into exosomes derived from mammalian cells or milk to achieve in vivo gene delivery and expression. This technology adopts readily available reagents and excipients that are recorded in the pharmacopoeias worldwide, simplifies the process of loading and preparation, reduces the cost, and is easy for large-scale production. It is compatible with existing biopharmaceutical production processes and significantly improves the loading efficiency of large-fragment DNA molecules, offering a new and efficient tool for gene therapy.

### SUMMARY OF THE INVENTION

The present disclosure provides a method for efficiently loading DNA into exosomes. The exosomes are derived from mammalian cells or milk. A highly compressed DNA-polycationic compression agent complex is formed by combining a polycationic compression agent with DNA, and then loaded into the membrane of the exosomes using physical measures, successfully achieving efficient loading of DNA as well as its in vivo gene delivery and expression.

The present disclosure provides a method for efficiently loading DNA into exosomes, wherein the exosomes are derived from mammalian cells or milk, and the method comprises steps of:
(1) thoroughly mixing a polycationic compression agent with DNA to obtain a polycationic-DNA complex,
(2) adding the polycationic-DNA complex into the exosomes derived from mammalian cells or milk and mixing well, and
(3) complete loading DNA.

Further, the method may comprise step (4) of removing unloaded DNA and debris.

Further, step (4) further comprises adding heparin to dissociate unloaded DNA-protamine complex, wherein the removal of unloaded free DNA is carried out by one or more method selected from the group consisting of salt active nuclease treatment, DNase I treatment, and Benzonase treatment, and the removal of nucleic acid debris can be carried out by method selected from anion exchange chromatography purification and/or Capto Core 700 chromatography purification.

Further, the method may comprise step (5) of verifying loading efficacy by qPCR method.

Further, in step (1), the exosomes derived from mammalian cells or milk are unmodified exosomes, and also can be engineered exosomes.

Further, the engineered exosomes are exosomes modified with syncytin-1.

Further, the engineered exosomes are exosomes modified with single-chain integrin αLβ2.

Further, the engineered exosomes are exosomes modified with single-chain integrin αDβ2.

Further, in step (1), a ratio of DNA to the exosomes is 3-20:1.

Further, the polycationic compression agent can be one or more selected from the group consisting of protamine, poly-lysine, poly-arginine, and polyethyleneimine (PEI).

Further, the polycationic compression agent can be protamine, and a ratio of protamine to DNA is 2-10:1.

Further, the polycationic compression agent can be protamine, and a ratio of protamine to DNA is 7:1.

Further, in step (2), after mixing protamine with exosomes and DNA, resulting precipitate from supernatant can be separated by centrifugation.

Further, in step (3), the loading of DNA can be carried out by ultrasonication method and/or thermal shock/cycling method.

Further, a cycle number of the thermal shock/cycling method is 1 to 20 cycles.

Further, a cycle number of the thermal shock/cycling method is 10 to 20 cycles.

Further, a thermal shock temperature of the thermal shock/cycling method can be 25 to 60 °C, and a cooling temperature can be 0 °C or 4 °C.

Further, the loading of the exosomes derived from milk is carried out with a thermal shock temperature of 60 °C, a cooling temperature of 0 °C, and a number of thermal shocks of 20; and the loading of the exosomes derived from cells is carried out with a thermal shock temperature of 60 °C.

Further, before DNA loading, pre-treating exosomes, such as adjusting pH.

Further, in step (3), adding an auxiliary molecule to facilitate DNA loading into exosome membrane, the auxiliary molecule is one or more selected from the group consisting of SM102, and DLin-MC3-DMA.

Further, the auxiliary molecule is added in a ratio of SM102/DLin-MC3-DMA:protamine:DNA=3:1:1 (w/w) or 6:2:1 (w/w).

Further, the selected auxiliary molecule is DLin-MC3-DMA, and a ratio of DNA to protamine is 1:2.

Further, DNA for loading has a size of 1.9 kbp to 9 kbp.

The present disclosure provides an exosome for efficiently loading DNA prepared by the method above, the exosome is derived from mammalian cells or milk.

The present disclosure provides use of the exosome for efficiently loading DNA prepared by the method above in the manufacture of a medicament.

Further, the medicament is used as a non-viral vector for in vivo DNA delivery in gene therapy.

The present disclosure provides a pharmaceutical composition comprising the exosome prepared by the method of the present disclosure.

The present disclosure further provides a method of gene therapy comprising administering the pharmaceutical composition of the present disclosure.

### Beneficial Effects:

(1) The method for efficiently loading DNA into exosomes provided by the present disclosure achieves efficient loading of a large-fragment DNA molecule, with an efficient loading range from 1.9 kbp to 9 kbp. This effectively solves the problem of low loading efficiency of a large-fragment DNA molecule in the field, thus enabling exosomes to carry a large-fragment target DNA when used as a tool for gene therapy. This expands the range of target genes available for gene therapy, offers greater drug development potential for exosomes, and provides a powerful tool for gene therapy.
(2) The present disclosure utilized one or more of protamine, poly-lysine, poly-arginine and polyethyleneimine to assist in the folding of DNA molecules for efficient loading. The above components are widely used drugs or reagents, resulting in prominent savings in experimental costs and even future treatment costs. The method achieves the difficult loading of a large-fragment DNA molecule in the most conventional loading manner, demonstrating notable efficiency and cost advantages in industrial applications.
(3) The present disclosure further innovatively incorporates one or more auxiliary factors, such as SM102 and DLin-MC3-DMA, into the loading of DNA into exosomes, thereby further enhancing the loading efficacy and stability. This provides strong support for applying the resulting loaded exosomes in gene therapy.
(4) The loaded exosomes in the present disclosure can be modified with syncytin-1 and/or the single-chain integrins αLβ2 and/or αDβ2, enabling specific targeting of inflamed vascular endothelial cells and podocytes in the glomeruli. This offers effective tools and directions for corresponding disease model research, drug development for relevant clinical diseases, and exploration of gene therapy methods.

### BRIEF DESCRIPTION OF THE DRAWINGS

FIG. 1: Plasmid map of mini-circle hRluc (1919 bp) of the present disclosure.

FIG. 2: Plasmid map of pRP-EGFP (3657 bp) vector of the present disclosure.

FIG. 3: Cytation5 imaging of EGFP expression after exosomes entry into cells in the present disclosure.

FIG. 4: Plasmid map of pRP-Nanoluc (4929 bp) of the present disclosure.

FIG. 5: Detection of NanoLuc enzyme activity in cell supernatant after incubation of exosomes loaded with DNA of the present disclosure with cells.

FIG. 6: Plasmid map of pSGLs-Nanoluc-pp-Fc (8929 bp) of the present disclosure.

FIG. 7: Detection of NanoLuc enzyme activity in cell supernatant after incubation of exosomes loaded with DNA of the present disclosure with cells.

FIG. 8: Detection of NanoLuc enzyme activity in cell supernatant after incubation of exosomes loaded with DNA of the present disclosure with cells.

FIG. 9: Detection of NanoLuc enzyme activity in cell supernatant of the present disclosure.

FIG. 10: Immunofluorescence detection of the targeting effect of exosomes of the present disclosure.

FIG. 11: Targeted exosome treatment resulting in Nanoluc expression in cells of the present disclosure.

### DETAILED DESCRIPTION OF THE EMBODIMENTS

Technical solutions of embodiments of the present disclosure are clearly and completely described below. Apparently, the embodiments described in the following are only some embodiments of the present disclosure, rather than all the embodiments. Any other embodiments obtained by those skilled in the art based on the embodiments in the present disclosure without any creative work fall within the scope of protection of the present disclosure.

### Example 1: Loading Low Molecular Weight Plasmid Mini-Circle hRluc DNA (1919 Bp) into Milk-Derived Exosomes, and Comparing the Effects of Various Polycations Such as Poly-Lysine, Poly-Arginine, Protamine and Polyethyleneimine (PEI) on DNA Loading

### 1. Methods:

(1) DNA Loading: The plasmid used for DNA loading was mini-circle hRluc DNA (plasmid vector, with a target fragment of hRluc and plasmid size of 1919 bp). 7.5E+12 milk-derived exosome particles was adjusted to pH 10.0 with 0.1 M Na₂CO₃/NaHCO₃, added with 100 μg of DNA at a ratio of DNA:exosomes=10:1 (copy number:particle number), mixed well, allowed to stand at 25 °C for 10 min, and then added with protamine (or poly-lysine, poly-arginine, polyethyleneimine (PEI)) at a ratio of polycation:DNA=5:1 (w/w), and mixed well. The resulting sample was subjected to ultrasound treatment (10 °C, 1200 w for 2 h). After that, the pH was adjusted to 8.0, and ultrasound treatment was continued for another 1 h. Following ultrasound treatment, heparin was added (protamine:heparin=1:3, w/w), and mixed well, and then treated with salt active nuclease with a final concentration of 5 U/mL at 37 °C for 1 h to digest free nucleic acids. All samples were purified through a Capto Core 700 chromatography column to remove free protamine-heparin-salt active nuclease. The components of different samples were further analyzed by purification using anion exchange chromatography column (buffer: 20 mM Tris pH 8.0 and 100 mM NaCl; elution buffer: 20 mM Tris pH 8.0 and 1 M NaCl; gradient elution method).

(2) Loading Verification: The loading efficacy was verified by qPCR assay.

### 2. Results:

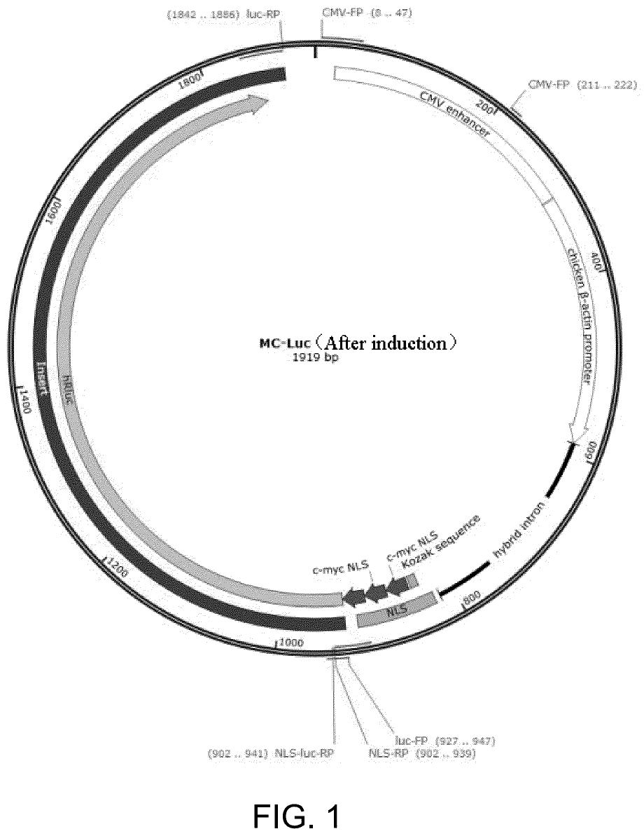

As shown in FIG. 1 and Table 1, the low-salt elution fractions from the anion exchange chromatography column contained peaks corresponding to the loaded exosomes. Each 100 exosome particles were found to load approximately 16-63 copies of the DNA plasmid, with the protamine group demonstrating the best loading efficacy. No loading of mini-circle hRluc DNA was detected in the experimental group without polycations.

TABLE 1
qPCR Results and Loading Capacity

| | | | | Plasmid |
|---|---|---|---|---|
| | | | | Copies |
| | | | qPCR Cq | per 100 |
| Sample Name and Anion | | | Value | Particles |
| Exchange Chromatography | | Particle | (Mean ± | (Mean ± |
| Purified Fraction | | Count | SD) | SD) |
| Exosomes + | Flow-through | 1.55E+10 | 28.40 ± 0.1 | 1 ± 2 |
| DNA + | Low-salt elution | 3.45E+10 | 13.79 ± 0.22 | 63 ± 8 |
| Protamine | High-salt elution | ND | 28.33 ± 0.17 | ND |
| Exosomes + | Flow-through | 1.75E+10 | 27.08 ± 0.34 | 2 ± 3 |
| DNA + | Low-salt elution | 4.01E+10 | 15.88 ± 0.11 | 46 ± 11 |
| Poly-lysine | High-salt elution | ND | 30.11 ± 0.54 | ND |
| Exosomes + | Flow-through | 1.20E+10 | 29.09 ± 0.46 | 1 ± 3 |
| DNA + | Low-salt elution | 3.28E+10 | 16.08 ± 0.12 | 35 ± 4 |
| Poly-arginine | High-salt elution | ND | 30.77 ± 0.63 | ND |
| Exosomes + | Flow-through | 1.77E+10 | 27.67 ± 0.43 | 1 ± 2 |
| DNA + PEI | Low-salt elution | 4.57E+10 | 18.54 ± 0.26 | 16 ± 6 |
| | High-salt elution | ND | 29.04 ± 0.30 | ND |
| Exosomes + | Flow-through | 1.02E+10 | 30.30 ± 0.42 | 1 ± 0.1 |
| DNA | Low-salt elution | 2.99E+10 | 35.26 ± 0.28 | 1 ± 0.5 |
| | High-salt elution | ND | 28.33 ± 0.39 | ND |

### Example 2: Loading Small Molecular Weight Plasmid Mini-Circle hRuc DNA (1919 bp) into Milk-Derived Exosomes and Verifying that Whether Protamine is a Key Reagent for DNA Loading

### 1. Methods:

(1) DNA Loading: The plasmid used for DNA loading was mini-circle hRluc DNA (plasmid vector, with a target fragment of hRluc and plasmid size of 1919 bp). 7.5E+12 milk-derived exosome particles was adjusted to pH 10.0 with 0.1 M Na₂CO₃/NaHCO₃, added with 100 μg of DNA at a ratio of DNA:exosomes=10:1 (copy number:particle number), and mixed well, allowed to stand at 25 °C for 10 min, then added with protamine at a ratio of protamine:DNA=5:1 (w/w), and mixed well. The resulting sample was subjected to ultrasound treatment (10 °C, 1200 w for 2 h). After that, the pH was adjusted to 8.0, and ultrasound treatment was continued for another 1 h. Following ultrasound treatment, heparin was added (protamine:heparin=1:3, w/w), mixed well, and then treated with salt active nuclease with a final concentration of 5 U/mL at 37 °C for 1 h to digest free nucleic acids. The samples were purified using Capto Core 700 chromatography and anion exchange chromatography to remove nucleic acid debris (buffer: 20 mM Tris pH 8.0 and 100 mM NaCl; elution buffer: 20 mM Tris pH 8.0 and 1 M NaCl; gradient elution method).

(2) Loading Verification: The loading efficacy was verified by qPCR assay.

### 2. Results:

As shown in Table 2, after DNA compression by protamine, each 100 exosome particles were found to load approximately 59 copies of the DNA plasmid, and no DNA loading was detected in the experimental group without protamine.

TABLE 2
qPCR Results and Loading Capacity

| | | | | Plasmid |
|---|---|---|---|---|
| | | | | Copies |
| | | | qPCR Cq | per 100 |
| Sample Name and Anion | | | Value | Particles |
| Exchange Chromatography | | Particle | (Mean ± | (Mean ± |
| Purified Fraction | | Count | SD) | SD) |
| Exosomes + | Flow-through | 1.36E+10 | 29.4 ± 0.03 | 0 ± 1 |
| DNA + | Low-salt | 3.52E+10 | 13.68 ± 0.05 | 59 ± 3 |
| Protamine | elution | | | |
| | High-salt | ND | 28.33 ± 0.2 | ND |
| | elution | | | |
| Exosomes + | Flow-through | 2.01E+10 | 29.3 ± 0.08 | 0 ± 1 |
| DNA | Low-salt | 4.43E+10 | 29.36 ± 1.5 | 2 ± 2 |
| | elution | | | |
| | High-salt | ND | 28.01 ± 0.01 | ND |
| | elution | | | |
| Exosomes + | Flow-through | 1.02E+10 | 30.3 ± 0.08 | 1 ± 1 |
| Protamine | Low-salt | 2.99E+10 | 30.26 ± 0.08 | 0 ± 1 |
| | elution | | | |
| | High-salt | ND | 29.33 ± 0.07 | ND |
| | elution | | | |
| DNA + | Flow-through | ND | 30.23 ± 0.17 | ND |
| Protamine | Low-salt | ND | 30.61 ± 0.15 | ND |
| | elution | | | |
| | High-salt | ND | 28.23 ± 0.11 | ND |
| | elution | | | |

### Example 3: Comparison of the Effects of Centrifugation Versus No Centrifugation on the Efficiency of Loading Mini-Circle hRluc DNA into Exosomes

Protamine, positively charged, can bind to negatively charged DNA through electrostatic actions, further compress DNA, and then change the loading efficiency of exosomes for DNA by controlling the loading method. Due to the significant precipitation that occurs during loading, the precipitate was dissolved by adding heparin after it formed, whether centrifugation was performed or not, followed by digestion of free nucleic acids with nuclease. Capto Core 700 was then used to remove free protamine, heparin, and nucleic acid debris from the system, and the effects of centrifugation versus no centrifugation on DNA loading efficacy were compared.

### 1. Methods:

(1) DNA Loading: The plasmid used for DNA loading was mini-circle hRluc DNA (plasmid vector, with a target fragment of hRluc and plasmid size of 1919 bp). The ratio of DNA to exosomes was set at 20:1 (copy number:particle number). Initially, 200 μL of OMEM was mixed with protamine and allowed to stand at 25 °C for 1 min, and 200 μL OMEM was mixed with 25 μg of DNA and allowed to stand at 25 °C for 1 min. Subsequently, the OMEM-DNA mixture was added to the OMEM+protamine mixture (protamine:DNA=7:1, w/w), vortexed for 1 min, and allowed to stand at 25 °C for 10 min. The OMEM-DNA-protamine mixture was added to milk-derived exosomes while mixing continuously. The sample was then placed in a metal bath at 37 °C with shaking at 120 rpm for 30 min, after which the sample was immediately placed in an ice-water mixture (4 °C) for 10 min; this constituted one cycle and was repeated 10 times. After completing the thermal shock treatment, the sample was equally divided into two portions. One portion was directly treated with heparin, mixed thoroughly, and then incubated with DNase I at 37 °C overnight for digestion. The other portion was centrifuged at 12,000 rpm for 20 min, and both the precipitate and supernatant were collected. The precipitate was washed three times with 1×PBS, re-dissolved in heparin (protamine:heparin=1:3 w/w), and adjusted to a final volume of 500 μL with 1×PBS before being incubated with DNase I at 37 °C overnight. The supernatant was directly mixed with heparin and DNase I and also incubated at 37 °C overnight. All samples were purified using Capto Core 700 chromatography to remove free protamine, heparin, and DNase I.

(2) The loading efficacy was verified by qPCR assay.

2. Results: After the 37-0 °C thermal-shock cycling treatment (37-0 °C), it was found that the impact of centrifugation on the Cq values was minimal, indicating that centrifugation was not necessary during the loading process, thus reducing the operational steps and time.

TABLE 3
qPCR Results and Loading Capacity

| | | | Plasmid Copies |
|---|---|---|---|
| | | qPCR Cq | per 100 |
| | Particle | Value | Particles |
| Sample Name | count | (Mean ± SD) | (Mean ± SD) |
| Milk-derived exosomes + | 1.58E+10 | 14.33 ± 0.16 | 64 ± 10 |
| DNA + Protamine - | | | |
| Centrifuged Precipitate | | | |
| Milk-derived exosomes + | 2.26E+10 | 16.23 ± 0.13 | 41 ± 5 |
| DNA + Protamine - | | | |
| Centrifuged Supernatant | | | |
| Milk-derived exosomes + | 3.34E+10 | 15.46 ± 0.18 | 52 ± 7 |
| DNA + Protamine - | | | |
| Not Centrifugation | | | |

### Example 4

Experimental Purpose: Loading Mini-Circle hRluc DNA into Milk-Derived Exosomes and Verifying the Effects of Different Ratios of Protamine to DNA and the Number of Thermal Shock Cycles on the Loading Efficiency

(1) DNA Loading: The plasmid used for DNA loading was mini-circle hRluc DNA (plasmid vector, with a target fragment of hRluc and plasmid size of 1919 bp). The ratio of DNA to exosomes was set at 20:1 (copy number:particle number). Initially, 200 μL of OMEM was mixed with protamine and allowed to stand for 1 min, and 200 μL OMEM was mixed with 10 μg of DNA and allowed to stand for 1 min. Subsequently, the OMEM-DNA mixture was added to the OMEM+protamine mixture (protamine:DNA=2:1, 5:1, 7:1, or 10:1, w/w), vortexed for 1 min, and allowed to stand at 25 °C for 10 min. The OMEM-DNA-protamine mixture was added to 2.5E+11 milk-derived exosomes, mixed, and incubated at 37 °C, 120 rpm for 30 min. The sample was then immediately placed in an ice-water bath (0 °C) and allowed to stand for 10 min (for sample groups, the steps from 37 °C to an ice-water bath were repeated 1-3 times). After centrifugation at 12000 rpm for 20 min, the precipitate was collected. The precipitate was washed twice with 1×PBS and then re-dissolved in heparin (protamine:heparin=1:3 w/w). The re-dissolved samples were adjusted to a final volume of 500 μL with 1×PBS. All samples were passed through Capto Core 700 to remove free protamine-heparin.

(2) Loading Verification: The loading efficacy was verified by qPCR assay.

(3) Expression Verification: The exosomes with the highest loading efficiency (DNA+protamine+milk-derived exosomes (DNA and protamine at a mass ratio of 1:10 group)+heparin−incubation for 2 cycles) were injected into the tail vein of mice, and heart, liver, spleen, lung, and kidney tissues were collected 4 h post-administration, ground, and centrifuged to obtain supernatants for Rluc enzyme activity detection.

### 2. Results

As shown in Tables 4-5, various ratios of DNA to protamine promoted the loading of nucleic acids into exosomes. The ratios ranged from 1:2 to 1:10 exhibited good loading efficacy, especially at 1:7. An increase in the number of thermal shock cycles improved the loading capacity of exosomes, with three cycles demonstrating significantly higher loading efficiency than one cycle. In vivo experiments, the expression of hRluc was detected and its enzymatic activity was confirmed, proving that exosomes loaded with the Rluc gene plasmid could transfect cells in vivo and achieve gene expression.

TABLE 4
qPCR Results and Loading Capacity

| | | qPCR Cq | Plasmid Copies |
|---|---|---|---|
| | Particle | Value | per 100 Particles |
| Sample Name | Count | (Mean ± SD) | (Mean ± SD) |
| Milk-derived Exosomes + DNA + Protamine + | 2.93E+07 | 13.67 ± 0.5 | 48 ± 10 |
| Heparin (1:2)- Incubation, 1 Cycle | | | |
| Milk-derived Exosomes + Heparin- | 3.27E+09 | 29.33 ± 0.9 | 0 ± 8 |
| Incubation, 1 Cycle | | | |
| DNA + Protamine (1:2)- Incubation, 1 Cycle | ND | 29.23 ± 0.8 | ND |
| Milk-derived Exosomes + Protamine - | 1.63E+09 | 30.26 ± 1.2 | 0 ± 6 |
| Incubation, 1 Cycle | | | |
| Milk-derived Exosomes + DNA- Incubation, | 1.33E+09 | 28.33 ± 0.3 | 0 ± 2 |
| 1 Cycle | | | |
| Milk-derived Exosomes + DNA + Protamine | 6.37E+08 | 10.46 ± 0.9 | 56 ± 8 |
| (1:2) + Heparin- Incubation, 2 Cycles | | | |
| Milk-derived Exosomes + DNA + Protamine | 1.13E+09 | 12.58 ± 0.9 | 63 ± 4 |
| (1:2) + Heparin- Incubation, 3 Cycles | | | |
| Milk-derived Exosomes + DNA + Protamine | 1.19E+10 | 15 ± 0.3 | 54 ± 8 |
| (1:5) + Heparin- Incubation, 1 Cycle | | | |
| Milk-derived Exosomes + Heparin- | 1.06E+10 | 30.36 ± 0.4 | 0 ± 2 |
| Incubation, 1 Cycle | | | |
| DNA + Protamine (1:5)- Incubation, 1 Cycle | ND | 33.08 ± 1.7 | ND |
| Milk-derived Exosomes + Protamine - | 6.04E+09 | 35.26 ± 0.5 | 0 ± 3 |
| Incubation, 1 Cycle | | | |
| Milk-derived Exosomes + DNA- Incubation, | 1.25E+10 | 32.5 ± 0.2 | 0 ± 2 |
| 1 Cycle | | | |
| Milk-derived Exosomes + DNA + Protamine | 1.15E+10 | 14.9 ± 0.7 | 59 ± 9 |
| (1:5) + Heparin- Incubation, 2 Cycles | | | |
| Milk-derived Exosomes + DNA + Protamine | 1.30E+10 | 14.25 ± 1.9 | 72 ± 13 |
| (1:5) + Heparin- Incubation, 3 Cycles | | | |
| Milk-derived Exosomes + DNA + Protamine | 1.42E+10 | 14.88 ± 1.6 | 71 ± 10 |
| (1:7) + Heparin- Incubation, 1 Cycle | | | |
| Milk-derived Exosomes + Heparin- | 1.53E+10 | 30.1 ± 0.3 | 0 ± 2 |
| Incubation, 1 Cycle | | | |
| DNA + Protamine (1:7)- Incubation, 1 Cycle | ND | 32.3 ± 0.4 | ND |
| Milk-derived Exosomes + Protamine - | 1.33E+10 | 34.62 ± 0.5 | 0 ± 3 |
| Incubation, 1 Cycle | | | |
| Milk-derived Exosomes + DNA- Incubation, | 1.23E+10 | 32.96 ± 0.2 | 0 ± 2 |
| 1 Cycle | | | |
| Milk-derived Exosomes + DNA + Protamine | 2.03E+10 | 12.32 ± 0.6 | 80 ± 8 |
| (1:7) + Heparin- Incubation, 2 Cycles | | | |
| Milk-derived Exosomes + DNA + Protamine | 1.86E+10 | 12.62 ± 0.2 | 89 ± 10 |
| (1:7) + Heparin- Incubation, 3 Cycles | | | |
| Milk-derived Exosomes + DNA + Protamine | 1.94E+10 | 13.04 ± 1.3 | 52 ± 11 |
| (1:10) + Heparin- Incubation, 1 Cycle | | | |
| Milk-derived Exosomes + Heparin- | 1.68E+10 | 29.66 ± 0.1 | 0 ± 2 |
| Incubation, 1 Cycle | | | |
| DNA + Protamine (1:10)- Incubation, 1 | ND | 34.27 ± 2.1 | ND |
| Cycle | | | |
| Milk-derived Exosomes + Protamine - | 1.47E+10 | 32.53 ± 0.3 | 0 ± 4 |
| Incubation, 1 Cycle | | | |
| Milk-derived Exosomes + DNA- Incubation, | 1.58E+10 | 34.43 ± 0.8 | 0 ± 8 |
| 1 Cycle | | | |
| Milk-derived Exosomes + DNA + Protamine | 1.19E+10 | 15 ± 0.3 | 63 ± 6 |
| (1:10) + Heparin- Incubation, 2 Cycles | | | |
| Milk-derived Exosomes + DNA + Protamine | 1.06E+10 | 30.36 ± 0.4 | 69 ± 8 |
| (1:10) + Heparin- Incubation, 3 Cycles | | | |

TABLE 5
Detection Results of RLuc Enzyme Activity
in Tissues Post Exosome Injection in Vivo

| Tissue Name | Replicate 1 | Replicate 2 | Replicate 3 | Mean | SD |
|---|---|---|---|---|---|
| Heart | 201 | 230 | 214 | 215 | 14.5 |
| Liver | 641 | 660 | 639 | 647 | 11.6 |
| Spleen | 189 | 193 | 176 | 186 | 8.9 |
| Lung | 200 | 209 | 189 | 199 | 10.0 |
| Kidney | 231 | 240 | 242 | 238 | 5.9 |
| Heart | 1004 | 1109 | 1120 | 1078 | 64.0 |

### Example 5: Loading Medium Molecular Weight Plasmid pRP-EGFP (3657 bp) into Cells and Exosomes at a Fixed Ratio of Protamine to DNA, and Verifying the Difference in Loading Efficiency Between Cell-Derived Exosomes and Milk-Derived Exosomes

### 1. Methods:

(1) DNA Loading: The plasmid used for DNA loading was pRP-EGFP DNA (expression vector, with a target fragment of EGFP and plasmid size of 3657 bp). The ratio of DNA to exosomes was set at 10:1 (copy number:particle number). 2 mL OMEM medium was mixed with protamine, and 2 mL OMEM was mixed with DNA. The OMEM-DNA mixture was then added to the OMEM-protamine mixture (protamine:DNA=7:1, w/w), vortexed for 1 min, and allowed to stand at 25 °C for 10 min. This mixture was added to the cell-derived exosomes or milk-derived exosomes and mixed. The samples were placed in an ice-water bath and allowed to stand for 30 min, followed by thermal shock at 42 °C for 90 s, and then stood on ice (0 °C) for 3 min; this constituted one cycle and was repeated 10 times. Heparin was then added to all samples (protamine:heparin=1:3 w/w). Each sample was equally divided into two portions. One portion was treated with DNase I (final concentration 0.1 mg/mL) and incubated overnight at 37 °C before purification through Capto Core 700, while the other portion was purified directly without enzymatic digestion.

(2) The particle counts of the loaded samples were detected by NanoFCM.

(3) The loading efficacy was verified by qPCR assay.

(4) Verification of Loaded Exosome Entry into Cells: The loaded samples were incubated with HepG2 cells, and after 72 hours of cell culture, fluorescence images were captured using a Cytation5 (Biotek) to detect protein expression following plasmid uptake.

The composition of the samples is shown in the table below.

| | pRP-EGFP DNA | Protamine | Exosomes | Heparin | |
|---|---|---|---|---|---|
| Sample Name | (μg) | (μg) | (particle) | (μg) | DNaseI |
| Cell-derived Exosomes | 152.6 | 1068.2 | 2.27E+12 | 3205 | — |
| Cell-derived | 152.6 | 1068.2 | 2.27E+12 | 3205 | 0.1 |
| Exosomes + DNaseI | | | | | mg/mL |
| Milk-derived Exosomes | 30 | 210 | 1.05E+12 | 630 | — |
| Milk-derived Exosomes + | 30 | 210 | 1.05E+12 | 630 | 0.1 |
| DNaseI | | | | | mg/mL |

### 2. Results:

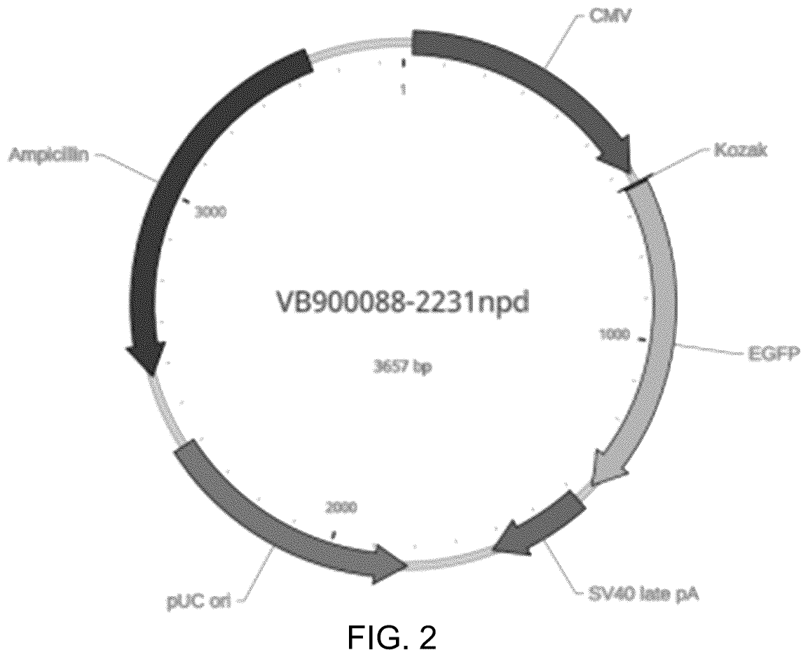

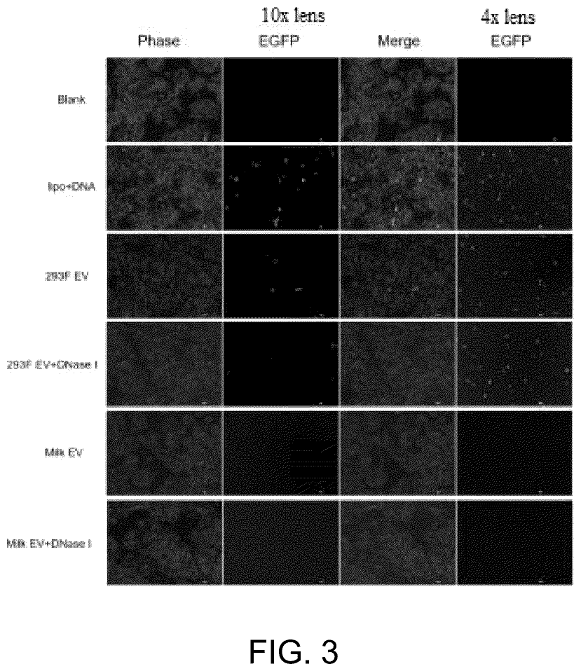

As shown in FIGS. 2-3 and Table 6, each 100 exosome particles were found to load approximately 20 plasmid copies on average, with cell-derived exosomes demonstrating higher loading efficiency. Due to the action of syncytin, the cell-derived exosomes loaded with DNA facilitated the fusion of exosome and cell membranes, allowing DNA to enter into the cell and be released, thereby avoiding lysosomal degradation. This resulted in significant green fluorescence after nuclear transcription and subsequent translation of EGFP. Conversely, the milk-derived exosomes loaded with the same amount of DNA did not show significant green fluorescence, likely due to most of the DNA being degraded by lysosomes after entry into the cell.

### Example 6: Loading Medium Molecular Weight Plasmid pRP-Nanoluc (4929 bp) into Cell-Derived or Milk-Derived Exosomes and Comparing the Effects of Different Thermal Shock Methods on Loading Efficiency in Exosomes from Different Sources

### 1. Methods:

(1) DNA Loading: The plasmid used for DNA loading was pRP-Nanoluc DNA (expression vector, with a target fragment of Nanoluc and plasmid size of 4929 bp). The ratio of DNA to exosomes was set at 3:1 (copy number:particle number). For each sample group, 2 mL OMEM was mixed with protamine and allowed to stand for 1 min, and 2 mL OMEM was mixed with 3 μg of DNA and allowed to stand at 25 °C for 1 min. The OMEM-DNA mixture was then added to the OMEM+protamine mixture (protamine:DNA=7:1, w/w), vortexed for 1 min, and allowed to stand at 25 °C for 10 min. This mixture was added to the cell-derived exosomes or milk-derived exosomes and mixed. This OMEM-DNA-protamine mixture was added to either cell-derived exosomes or milk-derived exosomes while mixing. Two thermal shock methods were employed: for samples treated at 60 °C, the samples were agitated at 120 rpm in a 60 °C shaker for 1 h, followed by cooling in an ice bath (0 °C) for 5 min; this constituted one cycle and was repeated three times. For samples treated at 42 °C, the samples were firstly placed in an ice-water bath (0 °C) for 30 min, then subjected to thermal shock at 42 °C for 90 s, followed by cooling in an ice-water bath (0 °C) for 3 min; this constituted one cycle and was repeated ten times. After thermal shock, all samples were mixed with heparin and vortexed at 25 °C for 1 min. Heparin was added to all the loaded samples (protamine:heparin=1:3 w/w). Each sample was then equally divided into two portions. One portion was digested overnight with DNase I at 37 °C before purification through Capto Core 700, while the other portion was purified directly through Capto Core 700 without enzymatic digestion.

(2) The particle counts of the loaded samples were detected by NanoFCM.

(3) The loading efficacy was verified by qPCR assay.

(4) Verification of Loaded Exosome Entry into Cells: The loaded samples were incubated with HepG2 cells, and after 72 hours of cell culture, the Nanoluc enzyme activity in the cell supernatant was detected.

The composition of the samples is shown in the table below.

| | Nanoluc-pp-Fc DNA | Protamine | Exosomes | Heparin | |
|---|---|---|---|---|---|
| Sample Name | (μg) | (μg) | (particle) | (μg) | Benzonase |
| Cell-derived Exosomes | 2.4 | 16.8 | 1.2E+11 | 50.4 | — |
| 60°C | | | | | |
| Cell-derived Exosomes + | 2.4 | 16.8 | 1.2E+11 | 50.4 | 5 U/mL |
| DNaseI 60°C | | | | | |
| Cell-derived Exosomes | 0.5 | 3.5 | 2.5E+10 | 10.5 | — |
| 42°C | | | | | |
| Cell-derived Exosomes + | 0.5 | 3.5 | 2.5E+10 | 10.5 | 5 U/mL |
| DNaseI 42°C | | | | | |
| Milk-derived Exosomes | 40 | 280 | 2.0E+12 | 840 | — |
| 60°C | | | | | |
| Milk-derived Exosomes + | 40 | 280 | 2.0E+12 | 840 | 5 U/mL |
| DNaseI 60°C | | | | | |
| Milk-derived Exosomes | 0.5 | 3.5 | 2.5E+10 | 10.5 | — |
| 42°C | | | | | |
| Milk-derived Exosomes + | 0.5 | 3.5 | 2.5E+10 | 10.5 | 5 U/mL |
| DNaseI 42°C | | | | | |

### 2. Results:

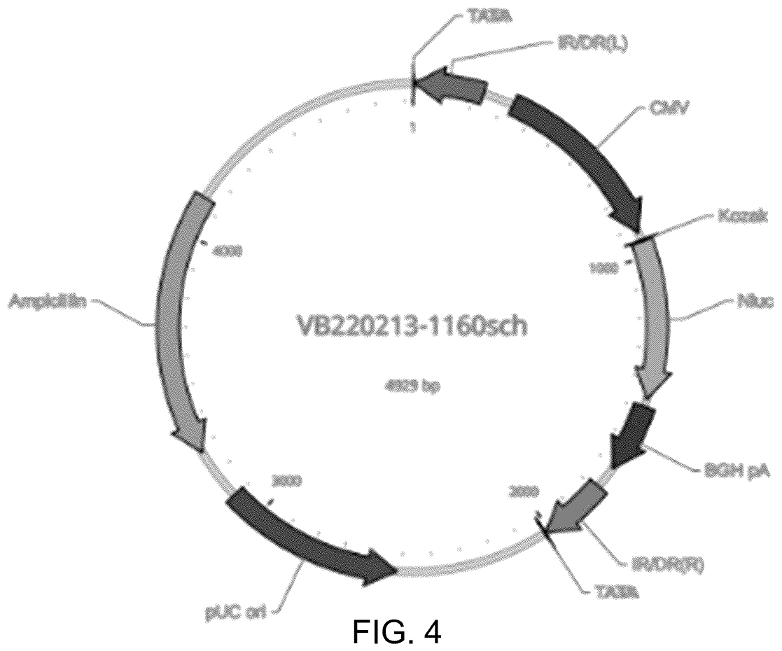

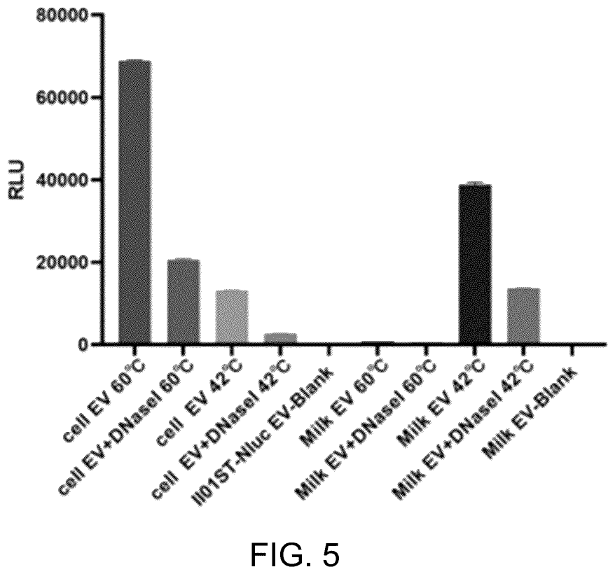

As shown in FIGS. 4-5 and Tables 7-8, each 100 exosome particles were found to load up to 50 plasmid copies on average. Under the condition of thermal shock at 60 °C, the loading capacity of pRP-Nanoluc plasmid was relatively higher in cell-derived exosomes, while under the condition of thermal shock at 42 °C, the loading capacity was higher in milk-derived exosomes. Additionally, successful expression and secretion of the functional Nanoluc protein were observed after DNA entry into cells, with detectable Nanoluc enzyme activity in the cell supernatant.

TABLE 7
Data related to loaded samples and loading capacity

| | | | Plasmid Copies |
|---|---|---|---|
| | | qPCR Cq | per 100 |
| | Particle | Value | Particles |
| Sample Name | Count | (Mean ± SD) | (Mean ± SD) |
| Cell-derived Exosomes | 2.12E+10 | 10.47 ± 0.44 | 51 ± 35 |
| 60°C | | | |
| Cell-derived Exosomes + | 1.33E+10 | 12.26 ± 0.36 | 40 ± 30 |
| DNaseI 60°C | | | |
| Cell-derived Exosomes | 5.19E+09 | 14.85 ± 0.23 | 37 ± 15 |
| 42°C | | | |
| Cell-derived Exosomes + | 3.44E+09 | 21.98 ± 0.07 | 16 ± 9 |
| DNaseI 42°C | | | |
| Cell-derived Exosomes − | 4.15E+09 | 26.30 ± 0.07 | 0 ± 2 |
| Blank | | | |
| Milk-derived Exosomes | 5.51E+11 | 16.27 ± 0.12 | 4 ± 8 |
| 60°C | | | |
| Milk-derived Exosomes + | 2.15E+11 | 15.05 ± 0.08 | 25 ± 6 |
| DNaseI 60°C | | | |
| Milk-derived Exosomes | 6.99E+09 | 14.19 ± 0.04 | 39 ± 15 |
| 42°C | | | |
| Milk-derived Exosomes + | 8.70E+09 | 14.89 ± 0.14 | 34 ± 4 |
| DNaseI 42°C | | | |
| Milk-derived Exosomes − | 7.99E+09 | 26.66 ± 0.15 | 0 ± 5 |
| Blank | | | |

TABLE 8
Data on Nanoluc enzyme activity in cell supernatant detected
after incubation of DNA-loaded exosomes with cells

| | Repli- | Repli- | Repli- | | |
|---|---|---|---|---|---|
| Sample Name | cate 1 | cate 2 | cate 3 | Mean | SD |
| Cell-derived | 68928 | 68821 | 69128 | 68959 | 156 |
| Exosomes 60°C | | | | | |
| Cell-derived | 20437 | 20929 | 20480 | 20615 | 272 |
| Exosomes + DNaseI | | | | | |
| 60°C | | | | | |
| Cell-derived | 13232 | 13230 | 13199 | 13220 | 19 |
| Exosomes 42°C | | | | | |
| Cell-derived | 2694 | 2737 | 2753 | 2728 | 31 |
| Exosomes + DNaseI | | | | | |
| 42°C | | | | | |
| Cell-derived | 281 | 272 | 260 | 271 | 11 |
| Exosomes − Blank | | | | | |
| Milk-derived | 768 | 774 | 783 | 775 | 8 |
| Exosomes 60°C | | | | | |
| Milk-derived | 569 | 582 | 550 | 567 | 16 |
| Exosomes + DNaseI | | | | | |
| 60°C | | | | | |
| Milk-derived | 39286 | 38216 | 39168 | 38890 | 587 |
| Exosomes 42°C | | | | | |
| Milk-derived | 13768 | 13689 | 13682 | 13713 | 48 |
| Exosomes + DNaseI | | | | | |
| 42°C | | | | | |
| Milk-derived | 244 | 255 | 258 | 252 | 7 |
| Exosomes − Blank | | | | | |

### Example 7: Verification of the Ability of Milk-Derived Exosomes to Successfully Load High Molecular Weight Plasmid pSGLs-Nanoluc-Pp-Fc (8929 bp) Using a Thermal Shock Method at 42 °C

### 1. Methods:

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 10:1 (copy number:particle number). For each sample group, 1 mL of OMEM medium was mixed with protamine, and 1 mL of OMEM medium was mixed with 8 μg of DNA. The OMEM-DNA mixture was then added to the OMEM-protamine mixture (protamine:DNA=7:1, w/w), vortexed for 1 min, and allowed to stand at 25 °C for 10 min. This mixture was added to the cell-derived exosomes or milk-derived exosomes, and mixed. This OMEM-DNA-protamine mixture was added to 8.80E+10 cell-derived exosomes. Subsequently, thermal shock was performed at 42 °C at 120 rpm for 1 h, followed by cooling in an ice-water bath (0 °C) for 5 min until samples were fully cooled; this constituted one cycle and was repeated three times. After thermal shock, all samples were mixed with heparin, and vortexed for 1 min. Heparin was added to all the loaded samples (protamine:heparin=1:3, w/w). Each sample was then equally divided into two portions: one was digested overnight with Benzonase nuclease at 37 °C before purification through Capto Core 700, while the other was purified through Capto Core 700 directly without enzymatic digestion.

(2) The particle counts of the loaded samples were detected by NanoFCM.

(3) The loading efficacy was verified by qPCR assay.

(4) Verification of Loaded Exosome Entry into Cells: The loaded samples were incubated with HepG2 cells, and after 72 hours of cell culture, the Nanoluc enzyme activity in the cell supernatant was detected.

### 2. Results:

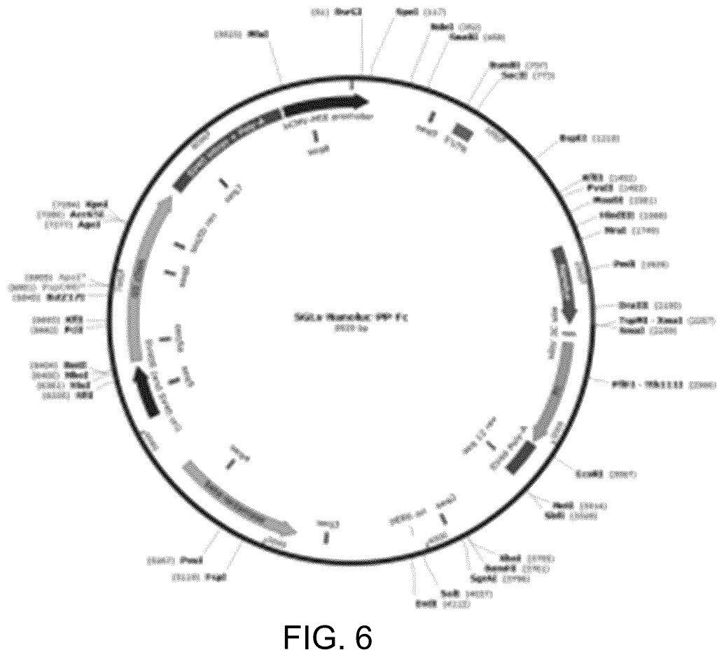

As shown in FIG. 6 and Table 9, under the condition of thermal shock at 42 °C for 1 h followed by ice bath at 0 °C for 5 min, repeated thermal shock three times, each 100 exosome particles were found to load approximately 20 plasmid copies. The addition of nuclease effectively removed the free DNA adhering to the surface of the exosomes.

TABLE 9
Data related to loaded samples and loading capacity

| | | | Plasmid Copies |
|---|---|---|---|
| | | | per 100 |
| | Particle | qPCR Cq Value | Particles |
| Sample Name | Count | (Mean ± SD) | (Mean ± SD) |
| Cell-derived Exosomes + | 4.53E+09 | 14.70 ± 0.42 | 35 ± 8 |
| DNA + Protamine + | | | |
| Heparin without | | | |
| digestion | | | |
| Cell-derived Exosomes + | 4.44E+09 | 15.23 ± 0.08 | 23 ± 2 |
| DNA + Protamine + | | | |
| Heparin with digestion | | | |

### Example 8: Loading High Molecular Weight Plasmid pSGLs-Nanoluc-Pp-Fc into Cell- and Milk-Derived Exosomes and Comparing the Effects of Different Thermal Shock Methods on DNA Loading Efficiency in Exosomes from Different Sources

### 1. Methods:

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 10:1 (copy number:particle number). For each sample group, 2 mL of OMEM medium was mixed with protamine, and 2 mL of OMEM medium was mixed with 6 μg of DNA. The OMEM-DNA mixture was then added to the OMEM-protamine mixture (protamine:DNA=7:1, w/w), vortexed for 1 min, and allowed to stand at 25 °C for 10 min. This mixture was added to the cell-derived exosomes or milk-derived exosomes, and mixed. This OMEM-DNA-protamine mixture was added to either 2.0E+11 cell-derived exosomes or 2.0E+11 milk-derived exosomes. The control group samples included all samples except for the exosomes. Two thermal shock methods were employed: for samples treated at 60 °C, the samples were agitated at 120 rpm in a 60 °C shaker for 1 h, followed by cooling in an ice-water bath (0 °C) for 5 min until samples were fully cooled; this constituted one cycle and was repeated three times. For samples treated at 42 °C, the samples were firstly placed in an ice-water bath (0 °C) for 30 min, then subjected to thermal shock at 42 °C for 90 s, followed by cooling in an ice-water bath (0 °C) for 3 min; this constituted one cycle and was repeated ten times. After thermal shock, all samples were mixed with heparin, and vortexed for 1 min. Heparin was added to all the loaded samples (protamine:heparin=1:3 w/w). Each sample was then equally divided into two portions. One portion was digested overnight with Benzonase nuclease at 37 °C before purification through Capto Core 700, while the other portion was purified directly through Capto Core 700 without enzymatic digestion.

(2) The particle counts of the loaded samples were detected by NanoFCM.

(3) The loading efficacy was verified by qPCR assay.

(4) Verification of Loaded Exosome Entry into Cells: The loaded samples were incubated with HepG2 cells, and after 72 hours of cell culture, the Nanoluc enzyme activity in the cell supernatant was detected.

### 2. Results:

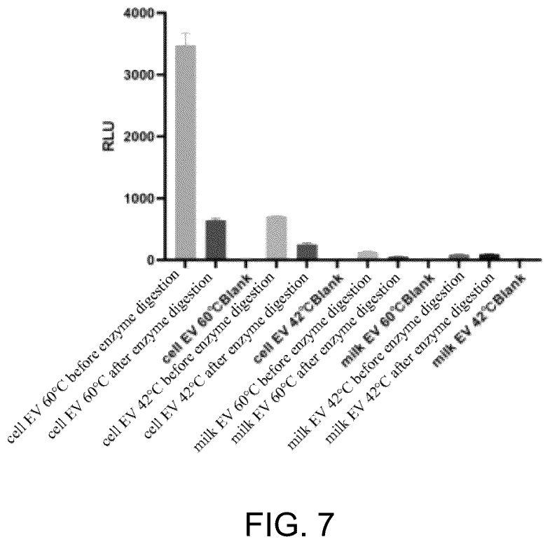

As shown in FIG. 7 and Tables 10-11, each 100 exosome particles were found to load approximately 2-40 plasmid copies, with the loading efficiency of cell-derived exosomes being higher than that of milk-derived exosomes, and the loading efficiency of cell-derived exosomes at 60 °C being superior to that at 42 °C.

TABLE 10
Data related to loaded samples and loading capacity

| | | | Plasmid Copies |
|---|---|---|---|
| | | | per 100 |
| | Particle | qPCR Cq Value | Particles |
| Sample Name | Count | (Mean ± SD) | (Mean ± SD) |
| Cell-derived Exosomes | 6.46E+10 | 10.9 ± 1.45 | 38 ± 7 |
| 60 °C, no digestion | | | |
| Cell-derived Exosomes | 9.50E+10 | 10.98 ± 1.15 | 28 ± 3 |
| 60 °C, digestion | | | |
| Cell-derived Exosomes | 1.35E+11 | 22.57 ± 0.21 | 0 ± 2 |
| 60 °C, Blank | | | |
| Cell-derived Exosomes | 6.05E+10 | 17.65 ± 0.87 | 3 ± 6 |
| 42 °C, no digestion | | | |
| Cell-derived Exosomes | 8.91E+10 | 19.9 ± 0.30 | 0 ± 5 |
| 42 °C, digestion | | | |
| Cell-derived Exosomes | 5.70E+10 | 23.23 ± 0.70 | 0 ± 7 |
| 42 °C, Blank | | | |
| Milk-derived Exosomes | 4.79E+10 | 16.57 ± 0.42 | 7 ± 3 |
| 60 °C, no digestion | | | |
| Milk-derived Exosomes | 3.98E+10 | 16.77 ± 0.70 | 2 ± 6 |
| 60 °C, digestion | | | |
| Milk-derived Exosomes | 4.68E+10 | 21.80 ± 0.22 | 0 ± 1 |
| 60 °C, Blank | | | |
| Milk-derived Exosomes | 2.13E+10 | 18.21 ± 0.19 | 4 ± 2 |
| 42 °C, no digestion | | | |
| Milk-derived Exosomes | 2.91E+10 | 15.66 ± 0.72 | 5 ± 5 |
| 42 °C, digestion | | | |
| Milk-derived Exosomes | 1.04E+10 | 23.17 ± 0.05 | 0 ± 1 |
| 42 °C, Blank | | | |

TABLE 11
Data on Nanoluc enzyme activity in cell supernatant detected
after incubation of DNA-loaded exosomes with cells

| | Repli- | Repli- | Repli- | | |
|---|---|---|---|---|---|
| Sample Name | cate 1 | cate 2 | cate 3 | Mean | SD |
| Cell-derived Exosomes | 3256 | 3606 | 3580 | 3481 | 195 |
| 60 °C, no digestion | | | | | |
| Cell-derived Exosomes | 668 | 614 | 657 | 646 | 29 |
| 60 °C, digestion | | | | | |
| Cell-derived Exosomes | 23 | 26 | 19 | 23 | 4 |
| 60 °C, Blank | | | | | |
| Cell-derived Exosomes | 707 | 719 | 722 | 716 | 8 |
| 42 °C, no digestion | | | | | |
| Cell-derived Exosomes | 239 | 239 | 281 | 253 | 24 |
| 42 °C, digestion | | | | | |
| Cell-derived Exosomes | 15 | 18 | 16 | 16 | 2 |
| 42 °C, Blank | | | | | |
| Milk-derived Exosomes | 137 | 129 | 148 | 138 | 10 |
| 60 °C, no digestion | | | | | |
| Milk-derived Exosomes | 56 | 54 | 63 | 58 | 5 |
| 60 °C, digestion | | | | | |
| Milk-derived Exosomes | 21 | 21 | 25 | 22 | 2 |
| 60 °C, Blank | | | | | |
| Milk-derived Exosomes | 98 | 88 | 96 | 94 | 5 |
| 42 °C, no digestion | | | | | |
| Milk-derived Exosomes | 94 | 102 | 98 | 98 | 4 |
| 42 °C, digestion | | | | | |
| Milk-derived Exosomes | 18 | 16 | 20 | 18 | 2 |
| 42 °C, Blank | | | | | |

### Example 9: Comparing Different Thermal Shock Temperatures and Times During the Loading Process in Order to Improve the Loading Efficiency of High Molecular Weight Plasmid pSGLs-Nanoluc-Pp-Fc into Exosomes and to Ensure that Nanoluc can Express and Exhibit Enzymatic Activity after Entry into Cells

### 1. Methods:

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 10:1 (copy number:particle number). 1 mL of OMEM medium was mixed with protamine, and DNA was subsequently added and mixed. The DNA-protamine mixture was then added to prepared milk-derived exosomes at pH 8.0, with continuous mixing (protamine:DNA=7:1, w/w). Water baths and shakers with different temperatures were preheated half an hour in advance. For samples subjected to one thermal shock at different temperatures, the samples were placed on ice for 10 min, heated at the corresponding temperature for 9 min, then agitated at 120 rpm on a preheated shaker for 175 min, and finally allowed to stand on ice for 5 min. For samples subjected to five thermal shocks at different temperatures, the samples were placed on ice for 10 min, heated at the corresponding temperature for 9 min, then agitated at 120 rpm on a preheated shaker for 31 min, and finally allowed to stand on ice for 5 min. The process was repeated five times. For samples subjected to ten thermal shocks at different temperatures, the samples were placed on ice for 10 min, heated at the corresponding temperature for 9 min, then agitated at 120 rpm on a preheated shaker for 13 min, and finally allowed to stand on ice for 5 min. The process was repeated ten times. For samples subjected to twenty thermal shocks at different temperatures, the samples were placed on ice for 10 min, heated at the corresponding temperature for 9 min, and finally allowed to stand on ice for 5 min. The process was repeated twenty times. After thermal shock, heparin was added to all samples, and mixed by inversion. All samples received 1 mM MgCl₂ and a final concentration of 5 U/mL Benzonase enzyme for overnight digestion at 37 °C. The digested samples were processed through Capto Core 700 to collect the flow-through and remove free protamine-heparin-Benzonase.

The composition of the samples is shown in the table below:

| | Nanoluc- | | | | |
|---|---|---|---|---|---|
| | pp-Fc | | | | |
| | DNA | Protamine | Exosomes | Heparin | |
| Sample Name | (μg) | (μg) | (particle) | (μg) | Benzonase |
| 25 °C-1 | 9.17 | 64.22 | 1.0E+11 | 192.66 | 5 U/mL |
| thermal shock | | | | | |
| 25 °C-5 | 9.17 | 64.22 | 1.0E+11 | 192.66 | 5 U/mL |
| thermal shocks | | | | | |
| 25 °C-10 | 9.17 | 64.22 | 1.0E+11 | 192.66 | 5 U/mL |
| thermal shocks | | | | | |
| 25 °C-20 | 9.17 | 64.22 | 1.0E+11 | 192.66 | 5 U/mL |
| thermal shocks | | | | | |
| 37 °C-1 | 9.17 | 64.22 | 1.0E+11 | 192.66 | 5 U/mL |
| thermal shock | | | | | |
| 37 °C- 5 | 9.17 | 64.22 | 1.0E+11 | 192.66 | 5 U/mL |
| thermal shocks | | | | | |
| 37 °C-10 | 9.17 | 64.22 | 1.0E+11 | 192.66 | 5 U/mL |
| thermal shocks | | | | | |
| 37 °C-20 | 9.17 | 64.22 | 1.0E+11 | 192.66 | 5 U/mL |
| thermal shocks | | | | | |
| 42 °C-1 | 9.17 | 64.22 | 1.0E+11 | 192.66 | 5 U/mL |
| thermal shock | | | | | |
| 42 °C-5 | 9.17 | 64.22 | 1.0E+11 | 192.66 | 5 U/mL |
| thermal shocks | | | | | |
| 42 °C- 10 | 9.17 | 64.22 | 1.0E+11 | 192.66 | 5 U/mL |
| thermal shocks | | | | | |
| 42 °C-20 | 9.17 | 64.22 | 1.0E+11 | 192.66 | 5 U/mL |
| thermal shocks | | | | | |
| 42 °C-3 | 9.17 | 64.22 | 1.0E+11 | 192.66 | — |
| thermal shocks | | | | | |
| 60 °C-1 | 9.17 | 64.22 | 1.0E+11 | 192.66 | 5 U/mL |
| thermal shock | | | | | |
| 60 °C-5 | 9.17 | 64.22 | 1.0E+11 | 192.66 | 5 U/mL |
| thermal shocks | | | | | |
| 60 °C-10 | 9.17 | 64.22 | 1.0E+11 | 192.66 | 5 U/mL |
| thermal shocks | | | | | |
| 60 °C-20 | 9.17 | 64.22 | 1.0E+11 | 192.66 | 5 U/mL |
| thermal shocks | | | | | |
| 42 °C-3 | 9.17 | 64.22 | 1.0E+11 | 192.66 | 5 U/mL |
| thermal shocks | | | | | |

(2) The particle counts of the loaded samples were detected by NanoFCM.

(3) The loading efficacy was verified by qPCR assay.

(4) Enzyme activity was detected using a microplate reader to verify Rluc expression after entry into cells.

### 2. Results:

As shown in Table 12, 20 thermal shocks for milk-derived exosomes at 60 °C can improve the loading capacity of high molecular weight DNA.

TABLE 12
qPCR Results and Loading Capacity

| | | | Plasmid Copies per |
|---|---|---|---|
| | Particle | qPCR Cq Value | 100 Particles |
| Sample Name | Count | (Mean ± SD) | (Mean ± SD) |
| 25 °C-1 thermal | 1.15E+11 | 15.71 ± 0.17 | 48 ± 2 |
| shock | | | |
| 25 °C-5 thermal | 1.24E+11 | 17.02 ± 0.73 | 18 ± 10 |
| shocks | | | |
| 25 °C-10 thermal | 7.86E+10 | 18.81 ± 0.17 | 8 ± 2 |
| shocks | | | |
| 25 °C- 20 thermal | 8.49E+10 | 17.62 ± 0.41 | 17 ± 4 |
| shocks | | | |
| 37 °C-1 thermal | 6.99E+10 | 16.97 ± 0.20 | 33 ± 3 |
| shock | | | |
| 37 °C-5 thermal | 7.44E+10 | 17.35 ± 0.13 | 23 ± 1 |
| shocks | | | |
| 37 °C-10 thermal | 5.07E+10 | 16.96 ± 0.27 | 45 ± 3 |
| shocks | | | |
| 37 °C- 20 thermal | 6.2E+10 | 16.94 ± 0.24 | 37 ± 3 |
| shocks | | | |
| 42 °C-1 thermal | 7.91E+10 | 17.65 ± 0.37 | 18 ± 4 |
| shock | | | |
| 42 °C-5 thermal | 6.39E+10 | 17.42 ± 0.33 | 26 ± 4 |
| shocks | | | |
| 42 °C-10 thermal | 8.27E+10 | 16.51 ± 0.59 | 38 ± 6 |
| shocks | | | |
| 42 °C-20 thermal | 4.92E+10 | 17.94 ± 0.13 | 23 ± 1 |
| shocks | | | |
| 42 °C-3 thermal | 8.42E+10 | 17.84 ± 0.48 | 15 ± 4 |
| shocks with | | | |
| digestion | | | |
| 60 °C-1 thermal | 8.57E+10 | 17.38 ± 0.21 | 20 ± 2 |
| shock | | | |
| 60 °C-5 thermal | 6.48E+10 | 16.34 ± 0.21 | 55 ± 2 |
| shocks | | | |
| 60 °C-10 thermal | 8.00E+10 | 16.40 ± 1.05 | 43 ± 1 |
| shocks | | | |
| 60 °C-20 thermal | 7.38E+10 | 15.79 ± 1.36 | 71 ± 16 |
| shocks | | | |

### Example 10: Loading SGLs-Nanoluc-Pp-Fc Plasmid into Cell-Derived Exosomes And Verifying that Whether the Addition of Different Auxiliary Molecules (Including Ionizable Lipid Molecules or CaCl₂)) During the Loading Process could Promote the Loading of DNA by Exosomes and the Effects of Different Ratios of Protamine to DNA on the Loading Efficiency of Exosomes Under the Addition of Auxiliary Molecules

### 1. Methods:

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 20:1 (copy number:particle number), with SM102/DLin-MC3-DMA:protamine:DNA=3:1:1 (w/w) or 6:2:1 (w/w). Prior to loading, the pH of the cell-derived exosomes was adjusted to 5.5. For the SM102 and DLin-MC3-DMA sample groups, 1 mL of OMEM medium was mixed with protamine and SM102/DLin-MC3-DMA, and DNA was added to the OMEM medium-protamine-SM102/DLin-MC3-DMA mixture. This mixture was then added to the cell-derived exosomes for thorough mixing. The sample group included OMEM medium, protamine, SM102/DLin-MC3-DMA, DNA, and exosomes, while the control group included all components except for the exosomes. For the CaCl₂ sample group, 1 mL of OMEM was mixed with protamine, and 2 mL of OMEM was mixed with 20 μg of DNA. The DNA was then added to the OMEM+protamine mixture (protamine:DNA=7:1, w/w). This mixture was mixed with 1E+11 cell-derived exosomes, followed by the addition of CaCl₂ to a final concentration of 20 mM. The sample group included OMEM medium, protamine, CaCl₂, DNA, and cell-derived exosomes, while the control groups included all components except for the exosomes. All samples were subjected to shaking at 120 rpm in a 42 °C shaker for 1 h, followed by cooling in an ice bath (0 °C) for 5 min until the samples were fully cooled; this constituted one cycle and was repeated three times. After that, the solution's pH was adjusted to neutral. After loading, heparin was added to all the loaded samples (protamine:heparin=1:3 w/w). Each sample was then equally divided into two portions. One portion was digested overnight with Benzonase nuclease at 37 °C before purification through Capto Core 700, while the other portion was purified directly through Capto Core 700 without enzymatic digestion.

(2) The particle counts of the loaded samples were detected by NanoFCM.

(3) The loading efficacy was verified by qPCR assay.

(4) Verification of Loaded Exosome Entry into Cells: The loaded samples were incubated with HepG2 cells, and after 72 hours of cell culture, the Nanoluc enzyme activity in the cell supernatant was detected.

### 2. Results:

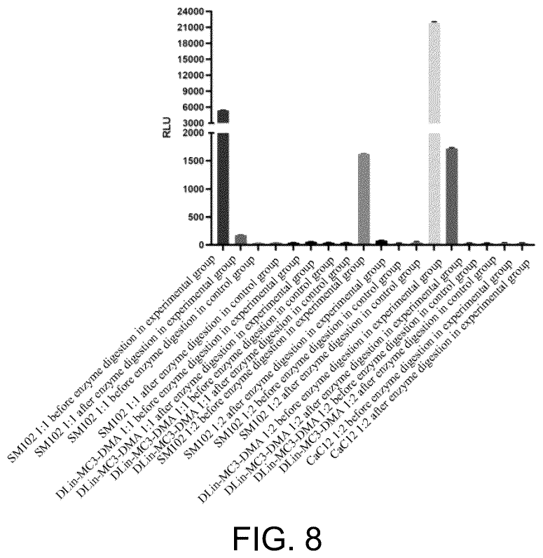

As shown in FIG. 8 and Tables 13-14, each 100 exosome particles were found to load approximately 1-60 plasmid copies, with the CaCl₂ group demonstrating relatively poor loading efficiency. Significant Nanoluc enzyme activity was detected in the cell supernatant of the SM102 and DLin-MC3-DMA groups, with the highest loading capacity observed in the DLin-MC3-DMA group when the ratio of DNA to protamine was 1:2.

TABLE 13
Data related to loaded samples and loading capacity

| | | | Plasmid Copies |
|---|---|---|---|
| | | qPCR | per 100 |
| | Particle | Cq Value | Particles |
| Sample Name | Count | (Mean ± SD) | (Mean ± SD) |
| SM102 1:1 Experimental | 6.20E+09 | 13.93 ± 0.2 | 171 ± 13 |
| Group Before Enzyme | | | |
| Digestion | | | |
| SM102 1:1 Experimental | 1.65E+10 | 15.68 ± 0.21 | 62 ± 10 |
| Group After Enzyme | | | |
| Digestion | | | |
| SM102 1:1 Control Group | ND | 22.66 ± 0.11 | ND |
| Before Enzyme Digestion | | | |
| SM102 1:1 Control Group | ND | 20.44 ± 0.23 | ND |
| After Enzyme Digestion | | | |
| DLin-MC3-DMA 1:1 | 8.00E+09 | 20.39 ± 0.05 | 2 ± 13 |
| Experimental Group | | | |
| Before Enzyme Digestion | | | |
| DLin-MC3-DMA 1:1 | 1.01E+10 | 20.42 ± 0.17 | 2 ± 10 |
| Experimental Group After | | | |
| Enzyme Digestion | | | |
| DLin-MC3-DMA 1:1 | ND | 26.10 ± 0.21 | ND |
| Control Group Before | | | |
| Enzyme Digestion | | | |
| DLin-MC3-DMA 1:1 | ND | 27.40 ± 0.30 | ND |
| Control Group After | | | |
| Enzyme Digestion | | | |
| SM102 1:2 Experimental | 3.60E+09 | 15.25 ± 0.30 | 50 ± 8 |
| Group Before Enzyme | | | |
| Digestion | | | |
| SM102 1:2 Experimental | 3.98E+09 | 17.30 ± 0.29 | 30 ± 6 |
| Group After Enzyme | | | |
| Digestion | | | |
| SM102 1:2 Control Group | ND | 23.95 ± 0.03 | ND |
| Before Enzyme Digestion | | | |
| SM102 1:2 Control Group | ND | 27.50 ± 0.05 | ND |
| After Enzyme Digestion | | | |
| DLin-MC3-DMA 1:2 | 4.80E+09 | 13.05 ± 0.13 | 72 ± 4 |
| Experimental Group | | | |
| Before Enzyme Digestion | | | |
| DLin-MC3-DMA 1:2 | 5.34E+09 | 14.80 ± 0.23 | 62 ± 8 |
| Experimental Group After | | | |
| Enzyme Digestion | | | |
| DLin-MC3-DMA 1:2 | ND | 20.12 ± 0.02 | ND |
| Control Group Before | | | |
| Enzyme Digestion | | | |
| DLin-MC3-DMA 1:2 | ND | 28.30 ± 0.05 | ND |
| Control Group After | | | |
| Enzyme Digestion | | | |
| CaCl2 1:2 Experimental | 1.82E+10 | 17.88 ± 0.08 | 86 ± 10 |
| Group Before Enzyme | | | |
| Digestion | | | |
| CaCl2 1:2 Experimental | 2.34E+10 | 20.17 ± 0.64 | 13 ± 2 |
| Group After Enzyme | | | |
| Digestion | | | |
| CaCl2 1:2 Control Group | 1.02 + 07 | 26.75 ± 0.30 | 0 ± 5 |
| Before Enzyme Digestion | | | |
| CaCl2 1:2 Control Group | 1.16 + 07 | 29.30 ± 0.19 | 0 ± 2 |
| After Enzyme Digestion | | | |

TABLE 14
Data on Nanoluc enzyme activity in cell supernatant detected
after incubation of DNA-loaded exosomes with cells

| Sample Name | Replicate 1 | Replicate 2 | Replicate 3 | Mean | SD |
|---|---|---|---|---|---|
| SM102 1:1 Experimental Group | 5328 | 5481 | 5452 | 5420 | 81 |
| Before Enzyme Digestion | | | | | |
| SM102 1:1 Experimental Group | 179 | 169 | 185 | 178 | 8 |
| After Enzyme Digestion | | | | | |
| SM102 1:1 Control Group Before | 39 | 35 | 39 | 38 | 2 |
| Enzyme Digestion | | | | | |
| SM102 1:1 Control Group After | 43 | 42 | 44 | 43 | 1 |
| Enzyme Digestion | | | | | |
| DLin-MC3-DMA 1:1 | 44 | 41 | 33 | 39 | 6 |
| Experimental Group Before | | | | | |
| Enzyme Digestion | | | | | |
| DLin-MC3-DMA 1:1 | 63 | 53 | 55 | 57 | 5 |
| Experimental Group After | | | | | |
| Enzyme Digestion | | | | | |
| DLin-MC3-DMA 1:1 Control | 46 | 42 | 35 | 41 | 6 |
| Group Before Enzyme Digestion | | | | | |
| DLin-MC3-DMA 1:1 Control | 41 | 38 | 46 | 42 | 4 |
| Group After Enzyme Digestion | | | | | |
| SM102 1:2 Experimental Group | 1628 | 1635 | 1612 | 1625 | 12 |
| Before Enzyme Digestion | | | | | |
| SM102 1:2 Experimental Group | 79 | 69 | 85 | 78 | 8 |
| After Enzyme Digestion | | | | | |
| SM102 1:2 Control Group Before | 38 | 27 | 24 | 30 | 7 |
| Enzyme Digestion | | | | | |
| SM102 1:2 Control Group After | 36 | 45 | 63 | 48 | 14 |
| Enzyme Digestion | | | | | |
| DLin-MC3-DMA 1:2 | 21838 | 22110 | 21773 | 21907 | 179 |
| Experimental Group Before | | | | | |
| Enzyme Digestion | | | | | |
| DLin-MC3-DMA 1:2 | 1725 | 1704 | 1735 | 1721 | 16 |
| Experimental Group After | | | | | |
| Enzyme Digestion | | | | | |
| DLin-MC3-DMA 1:2 Control | 36 | 39 | 34 | 36 | 3 |
| Group Before Enzyme Digestion | | | | | |
| DLin-MC3-DMA 1:2 Control | 35 | 38 | 25 | 33 | 7 |
| Group After Enzyme Digestion | | | | | |
| CaCl₂ 1:2 Experimental Group | 40 | 34 | 42 | 39 | 4 |
| Before Enzyme Digestion | | | | | |
| CaCl₂ 1:2 Experimental Group | 42 | 31 | 31 | 35 | 6 |
| After Enzyme Digestion | | | | | |
| CaCl₂ 1:2 Control Group Before | 39 | 41 | 37 | 39 | 2 |
| Enzyme Digestion | | | | | |
| CaCl₂ 1:2 Control Group After | 32 | 35 | 29 | 32 | 3 |
| Enzyme Digestion | | | | | |

* indicates that the ratio in experimental groups and control group was the ratio of DNA to protamine.

### Example 11: Loading SGLs-Nanoluc-Pp-Fc Plasmid DNA into Cell-Derived Exosomes, Ensuring that Nanoluc can Express and Exhibit Enzymatic Activity after Entry into Cells; Verifying Whether the Exosomal Endocytosis Capacity is Enhanced for Engineered Exosomes Expressing Syncytin-1

### 1. Methods

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 10:1 (copy number:particle number). Both 293F exosomes and 293F-Syncytin-1 exosomes were used for loading. 2 mL of OMEM medium was mixed with protamine, and 2 mL of OMEM medium was mixed with 3 μg of DNA. The OMEM-DNA mixture was then added to the OMEM-protamine mixture (protamine:DNA=7:1, w/w), vortexed for 1 min, and allowed to stand at 25 °C for 10 min. This mixture was added to either 1E+11 293F exosomes or 1E+11 293F-Syncytin-1 exosomes, and mixed. The samples were subjected to thermal shock at 42 °C at 120 rpm for 1 hour, followed by incubation on ice (4 °C) for 5 min; this constituted one cycle and was repeated three times. After that, heparin was added to all samples (protamine:heparin=1:3 w/w). Each sample was then equally divided into two portions. One portion was digested overnight with DNaseI (final concentration 5 U/mL) at 37 °C before purification through Capto Core 700, while the other portion was purified directly through Capto Core 700 without enzymatic digestion.

(2) The particle counts of the loaded samples were detected by NanoFCM.

(3) The loading efficacy was verified by qPCR assay.

(4) Verification of Loaded Exosome Entry into Cells: The successfully loaded samples were incubated with HepG2 cells, and after 6 h of culture, the medium was replaced to remove free plasmids, followed by qPCR analysis of the cells.

(5) Dectection of Nanoluc expression using a microplate reader: The cells were cultured for 72 h, and Nanoluc enzyme activity in the cell supertanant was detected.

### 2. Results:

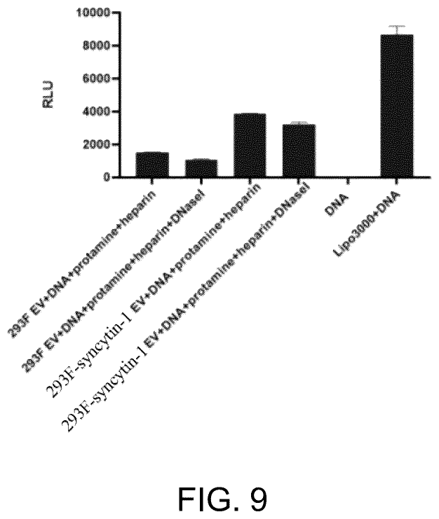

As shown in FIG. 9 and Tables 15-17, there was essentially no difference in DNA loading capacity between engineered exosomes and 293F wild-type exosomes, while engineered exosomes expressing Syncytin-1 demonstrated a stronger ability of entry into cells and higher Nanoluc expression levels.

TABLE 15
Data related to loaded samples and loading capacity

| | | | Plasmid Copies |
|---|---|---|---|
| | | qPCR Cq | per 100 |
| | Particle | Value | Particles |
| Sample Name | Count | (Mean ± SD) | (Mean ± SD) |
| 293F Exosomes + DNA + | 2.30E+10 | 15.42 ± 1.6 | 86 ± 11 |
| Protamine + Heparin | | | |
| 293F Exosomes + DNA + | 3.66E+10 | 17.56 ± 1.1 | 62 ± 9 |
| Protamine + Heparin + | | | |
| DNaseI | | | |
| 293F Exosomes + DNA + | 2.75E+10 | 25.33 ± 1.5 | 3 ± 3 |
| Heparin | | | |
| 293F Exosomes + DNA + | 2.69E+10 | 25.37 ± 1.0 | 4 ± 2 |
| Heparin + DNaseI | | | |
| 293F-Syncytin 1-Exosomes + | 3.22E+10 | 14.31 ± 0.6 | 79 ± 10 |
| DNA + Protamine + Heparin | | | |
| 293F-Syncytin 1-Exosomes + | 3.13E+10 | 18.58 ± 0.8 | 63 ± 12 |
| DNA + Protamine + | | | |
| Heparin + DNaseI | | | |
| 293F-Syncytin 1-Exosomes + | 3.13E+10 | 26..98 ± 1.3 | 2 ± 3 |
| DNA + Heparin | | | |
| 293F-Syncytin 1-Exosomes + | 2.95E+10 | 26.54 ± 1.2 | 2 ± 4 |
| DNA + Heparin + DNaseI | | | |
| 293F Exosomes | 2.30E+10 | 30.34 ± 1.5 | 0 ± 2 |
| 293F- Syncytin 1-Exosomes | 3.66E+10 | 31.58 ± 1.5 | 0 ± 3 |
| DNA | ND | 9.99 ± 0.9 | ND |
| DNA + DNaseI | ND | 28.96 ± 0.7 | ND |
| DNA + Protamine | ND | 26.3 ± 0.5 | ND |
| DNA + Protamine + DNaseI | ND | 27.53 ± 0.7 | ND |
| DNA + Protamine + Heparin | ND | 29.24 ± 1.3 | ND |
| DNA + Protamine + | ND | 30.21 ± 1.5 | ND |
| Heparin + DNaseI | | | |

TABLE 16
Results of cell entry ability of loaded exosomes

| | cells | qPCR_Cq | Copies per cell |
|---|---|---|---|
| samples | number | (Mean ± SD) | (Mean ± SD) |
| 293F Exosomes + DNA + | 1.00E+05 | 25.11 ± 0.8 | 14 ± 3 |
| Protamine + Heparin | | | |
| 293F Exosomes + DNA + | 1.00E+05 | 24.68 ± 0.7 | 12 ± 2 |
| Protamine + Heparin + | | | |
| DNaseI | | | |
| 293F-Syncytin 1-Exosomes + | 1.00E+05 | 21.12 ± 0.5 | 58 ± 5 |
| DNA + Protamine + Heparin | | | |
| 293F-Syncytin 1-Exosomes + | 1.00E+05 | 21.25 ± 0.3 | 58 ± 5 |
| DNA + Protamine + | | | |
| Heparin + DNaseI | | | |
| DNA | 1.00E+05 | 30.12 ± 1.5 | 0 ± 0.2 |
| Lipo3000 | 1.00E+05 | 17.55 ± 1.1 | 214 ± 10 |

TABLE 17
Data on Nanoluc enzyme activity detected in cell supernatant

| | Repli- | Repli- | Repli- | | |
|---|---|---|---|---|---|
| Sample Name | cate 1 | cate 2 | cate 3 | Mean | SD |
| 293F Exosomes + DNA + | 1766 | 1723 | 1760 | 1750 | 23 |
| Protamine + Heparin | | | | | |
| 293F Exosomes + DNA + | 1201 | 1143 | 1107 | 1150 | 47 |
| Protamine + Heparin + | | | | | |
| DNaseI | | | | | |
| 293F-Syncytin 1-Exosomes + | 3744 | 3720 | 3731 | 3732 | 12 |
| DNA + Protamine + Heparin | | | | | |
| 293F-Syncytin 1-Exosomes + | 3266 | 3501 | 3602 | 3456 | 172 |
| DNA + Protamine + | | | | | |
| Heparin + DNaseI | | | | | |
| DNA | 21 | 15 | 17 | 18 | 3 |
| Lipo3000 | 8433 | 8100 | 8814 | 8449 | 357 |

### Example 12: Exosomes Engineered to Express Single-Chain Integrin αLβ2 (or Single-Chain Integrin αDβ2) can Load the pSGLs-Nanoluc-Pp-Fc Plasmid and Specifically Target Inflamed Vascular Endothelial Cells and Podocytes in the Glomeruli

### 1. Methods:

(1) DNA Loading: The plasmid used for DNA loading was pSGLs-Nanoluc-pp-Fc DNA (expression vector, with a target fragment of Nanoluc and plasmid size of 8929 bp). The ratio of DNA to exosomes was set at 10:1 (copy number:particle number). For each sample group, 2 mL of OMEM medium was mixed with protamine, and 2 mL of OMEM medium was mixed with 3 μg of DNA. The OMEM-DNA mixture was then added to the OMEM+protamine mixture (protamine:DNA=7:1, w/w), vortexed for 1 min, and allowed to stand at 25 °C for 10 min. This OMEM-DNA-protamine mixture was added to 1E+11 engineered cell-derived exosomes. For the control group, DNA was loaded into wild-type 293F exosomes. The samples were subjected to thermal shock at 60 °C, and agitated at 120 rpm in a 60 °C shaker for 1 h, followed by an ice bath (0 °C) for 5 min until fully cooled; this constituted one cycle and was repeated three times. After thermal shock, all samples were mixed with heparin and vortexed for 1 min. Heparin was added to all the loaded samples (protamine:heparin=1:3 w/w). Each sample was digested overnight with Benzonase nuclease at 37 °C and then labeled with FITC-Integrinβ2 antibody before purification through Capto Core 700.

(2) The particle counts of the loaded samples were detected by NanoFCM.

(3) The loading efficacy was verified by qPCR assay.

(4) Immunofluorescence verification of exosome targeting ICAM-1-positive cells and podocytes: ICAM-1 positive cells and podocytes were seeded at 2E5 cells per well in a 24-well plate preloaded with glass slides. After 24 h, the slides were taken out and washed three times with PBS. They were fixed with 500 μL of paraformaldehyde for 10 min, and then washed three times with PBS, each for 5 min. 1 mL of 5% BSA/PBS buffer was added at room temperature for 2 h of blocking, and then washed three times with PBS, each for 5 min. Unlabeled wild-type EVs were added for blocking at a cell to EV ratio of 1:30000 for another 2 h.

In addition, engineered EVs loaded with single-chain integrin αLβ2 (or single-chain integrin αDβ2) and wild-type EVs were prepared, adjusted to a concentration of approximately 4E10 per mL, and mixed with an equal volume of PKH67 labeling buffer. The mixture was then incubated with 10 μL of PKH67 dye at room temperature in the dark for 30 min, followed by purification through core 700 to remove free dye. Then, 5E9 PKH67-labeled engineered EVs loaded with single-chain integrin αLβ2 (or single-chain integrin αDβ2) or wild-type EVs were added to the wells at 1 mL per slide, and incubated overnight at 4 °C in the dark. The slides were washed three times with PBST, each for 5 min. And then, the slides were taken out and mounted with a DAPI-containing mounting medium. After drying, the slides were imaged using a laser confocal microscope.

(5) ICAM-1 positive cells and podocytes were seeded at 2E5 cells per well in 24-well plates. After 24 h, wild-type EVs were added for blocking at a cell to EV ratio of 1:30000 at room temperature for 2 h. Subsequently, 5E9 engineered EVs loaded with single-chain integrin αLβ2 (or single-chain integrin αDβ2) and wild-type EVs (both blank and loaded with DNA) were added to the cells and incubated at 37 °C for 72 h. Cells were then collected for Nanoluc enzyme activity detection.

### Sequence Information:

SEQ. 1 (Amino acid sequence of Syncytin-1 (1-484 aa)):
MALPYHIFLFTVLLPSFTLTAPPPCRCMTSSSPYQEFLWRMQRPGNIDAPSYRSLSK
GTPTFTAHTHMPRNCYHSATLCMHANTHYWTGKMINPSCPGGLGVTVCWTYFTQ
TGMSDGGGVQDQAREKHVKEVISQLTRVHGTSSPYKGLDLSKLHETLRTHTRLVS
LFNTTLTGLHEVSAQNPTNCWICLPLNFRPYVSIPVPEQWNNFSTEINTTSVLVGPL
VSNLEITHTSNLTCVKFSNTTYTTNSQCIRWVTPPTQIVCLPSGIFFVCGTSAYRCLN
GSSESMCFLSFLVPPMTIYTEQDLYNYVISKPRNKRVPILPFVIGAGVLGALGTGIGG
ITTSTQFYYKLSQELNGDMERVADSLVTLQDQLNSLAAVVLQNRRALDLLTAERG
GTCLFLGEECCYYVNQSGIVTEKVKEIRDRIQRRAEELRNTGPWGLLSQWMPWILP
FLGPLAAIILLLLFGPCIFNLLVNFVSSRIEAVK
SEQ. 2 (Amino acid sequence of Integrin αLβ2):
Sequence Structure: SP(EWIF)-IntegrinαL(26-155)-(GGGGS)3-Integrinβ2(124-363)-
(GGGGS)3-IntegrinαL(338-612)-Fc-NPTN(Ig1-3)-TMD(EWIF)
MGRLASRPLLLALLSLALCRGRVVRYNLDVRGARSFSPPRAGRHFGYRVLQVGNG
VIVGAPGEGNSTGSLYQCQSGTGHCLPVTLRGSNYTSKYLGMTLATDPTDGSILAC
DPGLSRTCDQNTYLSGLCYLFRQNLQGPMLQGRPGFQECIKGNVGGGGSGGGGSG
GGGSGYPIDLYYLMDLSYSMLDDLRNVKKLGGDLLRALNEITESGRIGFGSFVDKT
VLPFVNTHPDKLRNPCPNKEKECQPPFAFRHVLKLINNSNQFQTEVGKQLISGNLD
APEGGLDAMMQVAACPEEIGWRNVTRLLVFATDDGFHFAGDGKLGAILTPNDGRC
HLEDNLYKRSNEFDYPSVGQLAHKLAENNIQPIFAVTSRMVKTYEKLTEIIPKSAVG
ELSEDSSNVVQLIKNAYNKLGGGGSGGGGSGGGGSSKQDLTSFNMELSSSGISADL
SRGHAVVGAVGAKDWAGGFLDLKADLQDDTFIGNEPLTPEVRAGYLGYTVTWLPS
RQKTSLLASGAPRYQHMGRVLLFQEPQGGGHWSQVQTIHGTQIGSYFGGELCGVD
VDQDGETELLLIGAPLFYGEQRGGRVFIYQRRQLGFEEVSELQGDPGYPLGRFGEAI
TALTDINGDGLVDVAVGAPLEEQGAVYIFNGRHGGLSPQPSQRIEGTQVLSGIQWFG
RSIHGVKDLEGDGLADVAVGAESQMIVLSSAESKYGPPCPPCPAPEAAGGPSVFLFP
PKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTV
DKSRWQEGNVFSCSVMHEALHNHYTQKSLSLSLGQNAGFVKSPMSETKLTGDAFE
LYCDVVGSPTPEIQWWYAEVNRAESFRQLWDGARKRRVTVNTAYGSNGVSVLRIT
RLTLEDSGTYECRASNDPKRNDLRQNPSITWIRAQATISVLQKPRIVTSEEVIIRDSPV
LPVTLQCNLTSSSHTLTYSYWTKNGVELSATRKNASNMEYRINKPRAEDSGEYHCV
YHFVSAPKANATIEVKAAPDITGHKRSENKNEGQDATMYCKSVGYPHPDWIWRK
KENGMPMDIVNTSGRFFIINKENYTELNIVNLQITEDPGEYECNATNAIGSASVVTL
LIGVNLLTVIGLWLFLILYC
SEQ. 3 (Amino acid sequence of Integrin αDβ2):
Sequence Structure: SP(EWIF)-αD(19-149)-(GGGGS)3β2(124-363)-(GGGGS)3-
αD(338-612)-Fc-NPTN(Ig1-3)-TMD(EWIF)
MGRLASRPLLLALLSLALCRGRVVRNLDVEEPTIFQEDAGGFGQSVVQFGGSRLVV
GAPLEVVAANQTGRLYDCAAATGMCQPIPLHIRPEAVNMSLGLTLAASTNGSRLLA
CGPTLHRVCGENSYSKGSCLLLGSRWEIIQTVPDATPECPHQEMGGGGSGGGGSGG
GGSGYPIDLYYLMDLSYSMLDDLRNVKKLGGDLLRALNEITESGRIGFGSFVDKTV
LPFVNTHPDKLRNPCPNKEKECQPPFAFRHVLKLTNNSNQFQTEVGKQLISGNLDA
PEGGLDAMMQVAACPEEIGWRNVTRLLVFATDDGFHFAGDGKLGAILTPNDGRCH
LEDNLYKRSNEFDYPSVGQLAHKLAENNIQPIFAVTSRMVKTYEKLTEIIPKSAVGEL
SEDSSNVVQLIKNAYNKLGGGGSGGGGSGGGGSQRASSSFQHEMSQEGESTALT
MDGLFLGAVGSFSWSGGAFLYPPNMSPTFINMSQENVDMRDSYLGYSTELALWKG
VQNLVLGAPRYQHTGKAVIF's
TQVSRQWRKKAEVTGTQIGSYFGASLCSVDVDSDGSTDLILIGAPHYYEQTRGGQ
VSVCPLRGRVQWQCDAVLRGEQGHPWGRFGAALTVLGDVNEDKLIDVAIGAPGE
QENRGAVYLFHGASESGISPSHSQRIASSQLSPRLQYFGQALSGGQDLTQDGLMDL
AVGARGQVLLLAESKYGPPCPPCPAPEAAGGPSVFLFPPKPKDTLMISRTPEVTCVV
VDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVM
HEALHNHYTQKSLSLSLGQNAGFVKSPMSETKLTGDAFELYCDVVGSPTPEIQWW
YAEVNRAESFRQLWDGARKRRVTVNTAYGSNGVSVLRITRLTLEDSGTYECRASN
DPKRNDLRQNPSITWIRAQATISVLQKPRIVTSEEVIIRDSPVLPVTLQCNLTSSSHTL
TYSYWTKNGVELSATRKNASNMEYRINKPRAEDSGEYHCVYHFVSAPKANATIEV
KAAPDITGHKRSENKNEGQDATMYCKSVGYPHPDWIWRKKENGMPMDIVNTSGR
FFIINKENYTELNIVNLQITEDPGEYECNATNAIGSASVVTLLIGVNLLTVIGLWLFLI
LYC

### 2. Results:

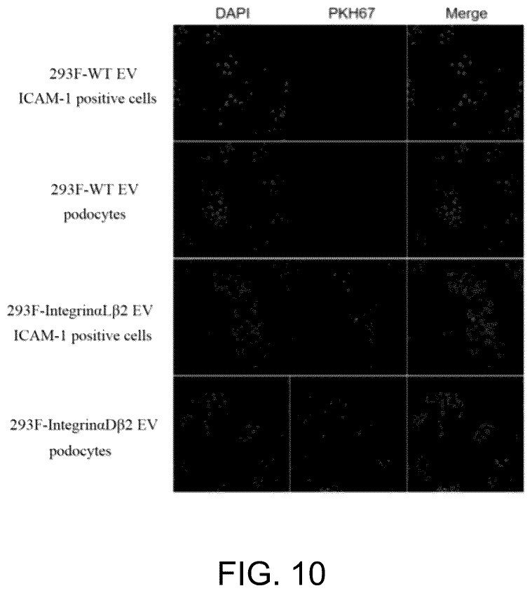

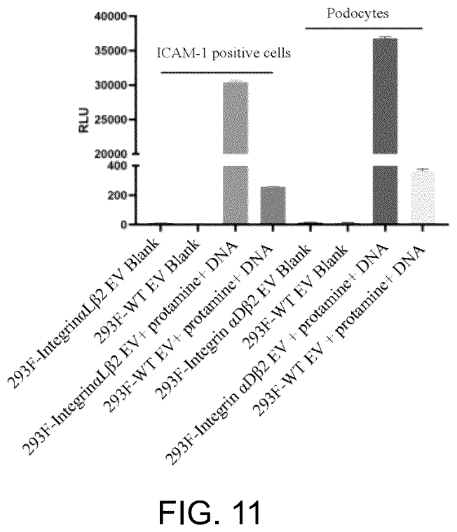

As shown in FIGS. 10-11 and Tables 18-19, the DNA loading capacity in engineered exosomes and wild-type exosomes were found to be comparable, with an average of 40-60 DNA copies loaded per 100 exosome particles. Immunofluorescence results indicated that engineered exosomes loaded with single-chain Integrin αLβ2 (or single-chain Integrin αDβ2) (green fluorescence) specifically bound to ICAM-1 positive cells and podocytes. Following the further loading of pSGLs-Nanoluc-pp-Fc DNA plasmid, Nanoluc enzyme activity assays demonstrated that the targeted engineered EVs could transport a greater number of loaded plasmids into the target cells and express more Nanoluc.

TABLE 18
Data related to loaded samples and loading capacity

| | | | Plasmid Copies |
|---|---|---|---|
| | | qPCR | per 100 |
| | Particle | Cq Value | Particles |
| Sample Name | Count | (Mean ± SD) | (Mean ± SD) |
| 293F-Integrin αDβ2 | 6.06E+10 | 30.11 ± 2.1 | 0 ± 2 |
| Exosomes (Blank) | | | |
| 293F-Integrin αLβ2 | 6.32E+10 | 31.22 ± 1.5 | 0 ± 3 |
| Exosomes (Blank) | | | |
| 293F-WT Exosomes Blank | 7.10E+10 | 29.56 ± 1.9 | 0 ± 1 |
| 293F-Integrin αDβ2 | 6.05E+10 | 19.32 ± 0.8 | 43 ± 13 |
| Exosomes + Protamine + | | | |
| DNA | | | |
| 293F-Integrin αLβ2 | 6.55E+10 | 18.79 ± 0.7 | 32 ± 10 |
| Exosomes + Protamine + | | | |
| DNA | | | |
| 293F-WT Exosomes + | 5.80E+10 | 18.85 ± 1.2 | 62 ± 10 |
| Protamine + DNA | | | |

TABLE 19
Data on Nanoluc enzyme activity detected in cells

| Cell | | | | | | |
|---|---|---|---|---|---|---|
| Name | Sample Name | Replicate 1 | Replicate 2 | Replicate 3 | Mean | SD |
| ICAM-1 | 293F-IntegrinαLβ2 | 10 | 12 | 13 | 11.7 | 1.5 |
| Positive | Exosomes (Blank) | | | | | |
| Cells | 293F-WT Exosomes | 9 | 7 | 10 | 8.7 | 1.5 |
| | (Blank) | | | | | |
| | 293F-IntegrinαLβ2 | 30687 | 30496 | 30047 | 30410.0 | 328.6 |
| | Exosomes + | | | | | |
| | Protamine + DNA | | | | | |
| | 293F-WT Exosomes + | 264 | 257 | 253 | 258.0 | 5.6 |
| | Protamine + DNA | | | | | |
| Podocytes | 293F-Integrin αDβ2 | 13 | 15 | 16 | 14.7 | 1.5 |
| | Exosomes (Blank) | | | | | |
| | 293F-WT Exosomes | 10 | 14 | 13 | 12.3 | 2.1 |
| | (Blank) | | | | | |
| | 293F-Integrin αDβ2 | 36787 | 37012 | 36598 | 36799.0 | 207.3 |
| | Exosomes + | | | | | |
| | Protamine + DNA | | | | | |
| | 293F-WT Exosomes + | 362 | 348 | 380 | 363.3 | 16.0 |
| | Protamine + DNA | | | | | |

To the claims:

1. A method for efficiently loading DNA into exosomes, wherein the exosomes are derived from mammalian cells or milk, the method comprises steps of:
(1) thoroughly mixing a polycationic compression agent with DNA to obtain a polycationic-DNA complex,
(2) adding the polycationic-DNA complex into the exosomes derived from mammalian cells or milk and mixing well, and
(3) loading the DNA.

2. The method for efficiently loading DNA into exosomes according to claim 1 further compriseing step (4) of removing unloaded DNA and debris,
adding heparin to dissociate the unloaded DNA-protamine complex, wherein removal of the unloaded free DNA is carried out by one or more method selected from the group consisting of salt active nuclease treatment, DNase I treatment, and Benzonase treatment, and removal of nucleic acid debris is carried out by method selected from anion exchange chromatography purification and/or Capto Core 700 chromatography purification.

3. (canceled)

4. The method for efficiently loading DNA into exosomes according to claim 1 further compriseing step (5) of verifying loading effect by quantitative polymerase chain reaction (qPCR) method.

5. The method for efficiently loading DNA into exosomes according to claim 2 wherein in the step (1), the exosomes derived from mammalian cells or milk are unmodified exosomes or engineered exosomes, and the engineered exosomes are exosomes modified with syncytin-1, exosomes modified with single-chain integrin αLβ2, or exosomes modified with single-chain integrin αDβ2.

6. (canceled)

7. (canceled)

8. (canceled)

9. The method for efficiently loading DNA into exosomes according to claim 5, wherein in the step (1), a ratio of the DNA to the exosomes is 3-20:1.

10. The method for efficiently loading DNA into exosomes according to claim 1, wherein in the step (2), the polycationic compression agent is one or more selected from the group consisting of protamine, poly-lysine, poly-arginine, and polyethyleneimine.

11. The method for efficiently loading DNA into exosomes according to claim 10, wherein the polycationic compression agent is protamine, and a ratio of protamine to the DNA is 2-10:1.

12. The method for efficiently loading DNA into exosomes according to claim 11, wherein the polycationic compression agent is protamine, and a ratio of protamine to the DNA is 7:1.

13. The method for efficiently loading DNA into exosomes according to claim 1, wherein the step (2) further comprises, after mixing protamine with the exosomes and the DNA, separating resulting precipitate from supernatant by centrifugation.

14. The method for efficiently loading DNA into exosomes according to claim 1, wherein in the step (3), the loading of the DNA is carried out by ultrasonication method and/or thermal shock/cycling method, and the thermal shock/cycling method is carried out with 1 to 20 cycles.

15. (canceled)

16. The method for efficiently loading DNA into exosomes according to claim 14, wherein the thermal shock/cycling method is carried with 10 to 20 cycles.

17. The method for efficiently loading DNA into exosomes according to claim 16, wherein the thermal shock/cycling method is carried with a thermal shock temperature of 25 to 60 °C, and a cooling temperature can be 0 °C or 4 °C.

18. The method for efficiently loading DNA into exosomes according to claim 17, wherein the loading of the exosomes derived from milk is carried out with the thermal shock temperature of 60 °C, the cooling temperature of 0 °C, and a number of thermal shocks of 20; and the loading of the exosomes derived from cells is carried out with the thermal shock temperature of 60 °C.

19. The method for efficiently loading DNA into exosomes according to claim 1, wherein before DNA loading, a step of pre-treating the exosomes is adjusting pH.

20. The method for efficiently loading DNA into exosomes according to claim 1, wherein in the step (3), further comprises adding an auxiliary molecule to facilitate the DNA loading into exosome membrane, wherein the auxiliary molecule is one or two selected from the group consisting of SM102, and DLin-MC3-DMA.

21. The method for efficiently loading DNA into exosomes according to claim 20, wherein the auxiliary molecule is added in a ratio of SM102/DLin-MC3-DMA:protamine:DNA-3:1:1 (w/w) or 6:2:1 (w/w).

22. The method for efficiently loading DNA into exosomes according to claim 21, wherein the auxiliary molecule is DLin-MC3-DMA, and a ratio of the DNA to protamine is 1:2.

23. The method for efficiently loading DNA into exosomes according to claim 1, wherein a size of the DNA for loading is 1.9 kbp to 9 kbp.

24. (canceled)

25. (canceled)

26. (canceled)

27. A pharmaceutical composition, comprising an exosome for efficiently loading DNA prepared by a method for efficiently loading DNA into exosomes, wherein the exosomes are derived from mammalian cells or milk, and the method comprises steps of:
(1) thoroughly mixing a polycationic compression agent with DNA to obtain a polycationic-DNA complex,
(2) adding the polycationic-DNA complex into the exosomes derived from mammalian cells or milk and mixing well, and
(3) loading the DNA.

28. A method of gene therapy, comprising administering a pharmaceutical composition,
wherein the pharmaceutical composition comprises an exosome for efficiently loading DNA prepared by a method for efficiently loading DNA into exosomes, wherein the exosomes are derived from mammalian cells or milk, and the method comprises steps of:
(1) thoroughly mixing a polycationic compression agent with DNA to obtain a polycationic-DNA complex,
(2) adding the polycationic-DNA complex into the exosomes derived from mammalian cells or milk and mixing well, and
(3) loading the DNA.
